(19) United States
(12) Patent Application Publication
(10) Pub. No.: US 2021/0364530 A1
(43) Pub. Date: Nov. 25, 2021

(54) DOUBLE-MULTIPLEX ASSAY FOR MULTIPLE IMMUNOGLOBULIN ISOTYPES

(71) Applicant: Aditx Therapeutics Inc., Mountain View, CA (US)

(72) Inventors: Ge Chen, Mountain View, CA (US); Shahrokh Shabahang, Mountain View, CA (US); Hong Liu, Mountain View, CA (US)

(21) Appl. No.: 17/323,990

(22) Filed: May 18, 2021

Related U.S. Application Data
(60) Provisional application No. 63/117,400, filed on Nov. 23, 2020.
(60) Provisional application No. 63/027,102, filed on May 19, 2020.

(51) Int. Cl. G01N 33/68

(52) U.S. Cl. CPC G01N 33/6854

(57) ABSTRACT

The present disclosure provides methods for assaying antibodies and related compositions, systems, and kits. More specifically, the disclosure relates to double-multiplex assays that detect multiple immunoglobulin isotypes against multiple different antigens simultaneously. The double-multiplex assay may be conducted using a single test sample.

### TECHNICAL FIELD

The present disclosure provides methods for assaying antibodies and related compositions, systems, and kits. More specifically, the disclosure relates to double-multiplex assays that detect multiple immunoglobulin isotypes against multiple different antigens simultaneously. The double-multiplex assay may be conducted using a single test sample.

### BACKGROUND

Currently, most antibody or immunoglobulin testing is performed in separate reactions for each isotype and against a single antigen at a time. This process requires multiple reactions for detection of antibodies of more than one isotype or against more than one. Current tests for detection of antibodies are primarily based on ELISA (enzyme-linked immunosorbent assay) or LFA (lateral flow assay) platforms, which are relatively expensive and time-consuming to carry out, especially if detection of multiple immunoglobulin isotypes or antibodies against multiple antigens is desired. Other assays, such as bead-based platforms sold by Luminex, are single-multiplex and allow for detection of antibodies against multiple antigens, but either do not distinguish between immunoglobulin isotypes or only allow for detection of one immunoglobulin isotype at a time.

### BRIEF SUMMARY

The present disclosure, according to one embodiment, provides a double-multiplex assay method for detecting at least two isotypes of antibodies against at least two antigens in a test sample. The method includes combining a test sample containing test antibodies with a mixture of at least two types of identifiably labelled microparticles, wherein each type of identifiably labelled microparticles is conjugated to a different antigen, to form microparticle-immunoglobulin complexes with test antibodies that specifically bind the antigens. The method next includes combining the microparticle-immunoglobulin complexes with detectably labelled anti-Ig-isotype antibodies against at least two different immunoglobulin isotypes to form microparticle-immunoglobulin-anti-Ig-isotype complexes. The method additionally includes, detecting identifiably labelled microparticle type and anti-Ig-isotype antibody type for the microparticle-immunoglobulin-anti-Ig-isotype complexes to generate detection data. The method further includes combining or analyzing detection data to generate at least four distinct data points, each data point corresponding to a different combination of test antibody isotype and antigen specificity. The method also includes using the data points to determine a test sample property.

The disclosure provides a more specific embodiments having one or more the following additional features, which may be combined with one another and with other elements of the present specification, including the example.

The different antigens may be from a single biological source and the test sample property may be whether the subject is positive or negative for antibodies against the biological source.

At least three different antigens may be conjugated to at least three types of identifiably labelled microparticles and detectably labelled anti-Ig-isotype antibodies against at against least three different immunoglobulin isotypes may be used to generate at least nine distinct types of data points.

The test sample may be from a human subject.

The test sample may have a volume of 0.1-20.0 μL.

The test sample may be whole blood, serum, plasma, nasal secretions, sputum, bronchial lavage, urine, stool, or saliva, particularly whole blood, serum, or plasma, and more specifically the whole blood, serum, or plasma obtained by finger-stick.

The test sample may be diluted prior to combining with mixture of at least two types of identifiably labelled microparticles. More specifically, the diluted biological sample may have a volume of 20-50 dl.

The identifiably labelled microparticles may be microspheres.

The microparticles may have a cross-section that is from 0.001 μm to 1000 μm in length.

The identifiably labelled microparticles may be identifiable by size, magnetic properties, fluorescence, ultraviolet-excited fluorescence wavelength, violet-excited fluorescence wavelength, fluorescence intensity, metal isotopes, or any combination thereof.

The detectably labelled anti-Ig-isotype antibodies may be identifiable by fluorescence properties, luminescent properties, or colorimetric properties or any combinations thereof.

The anti-Ig-isotype antibodies may include antibodies against IgG, IgM, IgA, or any combinations thereof and, more specifically, the antigens may be from a virus, bacteria, transplanted organ or tissue, tumor, or cancer.

The anti-Ig-isotype antibodies may include antibodies against IgG subtypes, and, more specifically, the antigens may be from a virus, bacteria, transplanted organ or tissue, tumor, or cancer

The anti-Ig-isotype antibodies may include antibodies against IgE subtypes and, more specifically, the antigens may be from an allergen.

The microparticle-immunoglobulin complexes may be combined with a mixture of the detectably labelled anti-Ig-isotype antibodies.

Alternatively, the microparticle-immunoglobulin complexes may be combined with each type of the detectably labelled anti-Ig-isotype antibodies separately in sequential steps or the microparticle-immunoglobulin complexes may be combined with sub-mixtures of some but not all of the anti-Ig-isotype antibodies separately in sequential steps, with one step per sub-mixture.

The detecting step may be carried out using flow cytometry or mass cytometry.

The first combining through generating data point steps may be carried out in a period of time of about 30 minutes to 3 hours.

The method may further include determining at least one indicator of accuracy for each data point, wherein the indicator of accuracy is sensitivity, specificity, concordance (correlation), positive predictive value, negative predictive value, false positive rate, or false negative rate. The test sample property may be positivity or negativity of the test sample for test antibodies of a specific antibody isotype, and positivity or negativity may be determined by concordance of data points for the antibody isotype against all antigens.

Alternatively or in addition, the test sample property may be positivity or negativity of the test sample for test antibodies against a specific antigen, and positivity or negativity may be determined by concordance of data points for antibodies against the antigen for all antibody isotypes.

The method may further include determining at least one indicator of accuracy for the test sample property, wherein the indicator of accuracy is sensitivity, specificity, concordance (correlation), positive predictive value, negative predictive value, false positive rate, or false negative rate.

The specificity of the test sample property may be increased without a decrease in sensitivity as compared to a corresponding assay that uses only a single type of data point to determine the test sample property.

Alternatively or in addition, the specificity of the test sample property may be increased at least ten fold as compared to a corresponding assay that uses only a single type of data point to determine the test sample property.

The present disclosure, in another embodiment, further provides a system for double-multiplexed assay of a test sample for at least two isotypes of antibodies against at least two antigens. The system includes at least two types of identifiably labelled microparticles conjugated to at least two antigens, wherein each type of identifiably labelled microparticle is conjugated to a different antigen, at least two types of microparticle-immunoglobulin complexes, wherein each type of microparticle-immunoglobulin complex includes an identifiably labelled microparticle conjugated to an antigen and a test antibody from the test sample specifically bound to the antigen, and at least two types of microparticle-immunoglobulin-anti-Ig-isotype complexes, wherein each type of microparticle-immunoglobulin-anti-Ig-isotype complex includes an identifiably labelled microparticle conjugated to an antigen, a test antibody from the test sample specifically bound to the antigen, and at least one detectably labelled anti-Ig-isotype antibody bound to the test antibody.

In a more specific embodiment of the system, each type of microparticle-immunoglobulin-anti-Ig-isotype complex includes at least two types of detectably labelled anti-Ig-isotype antibodies bound to the test antibodies.

The system may be operable to perform any of the above methods or any other methods disclosed herein and may include any compositions disclosed herein.

The disclosure also provides, in a further embodiment, a kit for double-multiplexed assay of a test sample for at least two isotypes of antibodies against at least two antigens. The kit includes one or more types of identifiably labelled microparticles, wherein each type of microparticle is conjugated to a different antigen, and two or more types of detectably labelled anti-Ig-isotype antibodies, wherein each type of anti-Ig-isotype antibody binds a different immunoglobulin isotype or subtype. The kit may further include instructions for use according to any of the above methods or any other methods disclosed herein or to form any of the above systems or any other systems or compositions disclosed herein.

### BRIEF DESCRIPTION OF THE DRAWINGS

FIG. 1 is a flow chart of an exemplary double-multiplex assay according to the present disclosure.

FIG. 2 is a schematic diagram of materials usable in a double-multiplex assay.

FIG. 3 depicts Median Fluorescence Intensity (MFI) measurements obtained using a comparative single-multiplex assay for three immunoglobulin isotypes (IgG, IgM, and IgA) against one SARS-CoV-2 antigen, the receptor binding domain (RBD) of the viral spike (S) protein in a SARS-CoV-2 exposure negative test sample and a SARS-CoV-2 positive test sample. Three individual samples are shown corresponding to three immunoglobulin isotypes.

FIG. 4 depicts a comparison of assay sensitivity between an ELISA and a double-multiplex assay as described herein (DM-Ab). The signal-to-noise ratio (S/N) is quantified in a double-multiplex assay for three immunoglobulin isotypes (IgG, IgM, and IgA) against each of three SARS-CoV-2 antigens (spike protein S1 (S1), RBD, and nucleoprotein (NP)).

FIG. 5 depicts an exemplary report including information determined by a double-multiplex assay of the present disclosure.

### DETAILED DESCRIPTION

The present disclosure provides methods for assaying antibodies and related compositions, systems and kits. More specifically, the disclosure relates to double-multiplex assays that detect multiple immunoglobulin isotypes against multiple different antigens simultaneously to provide distinct types of data points for different antigen and immunoglobulin isotype combinations. The double-multiplex assay may be conducted using a single test sample from a subject in a single assay. The double-multiplex assay may provide information regarding a test sample property using the data points.

In a specific embodiment, the different antigens are from a single biological source and the test sample property is whether the subject is positive or negative for antibodies against the biological source.

Information regarding a test sample property may then further be used to diagnose the subject. For example, it may be used to determine if the subject has been previously exposed to an infectious agent associated with at least two of the different antigens or, if so, days post exposure, whether a robust immune response has resulted, whether a protective immune response has resulted, whether there have likely been multiple exposures, whether the infectious agent has resulted in an actual infection of the subject, or if so, whether the infection is current, the stage or severity of infection, whether the infection has been resolved, or how long it has been since the infection was resolved.

In addition, test sample properties collected from different test samples from the same subject, whether of the same type of different types, concurrently or over time may also be used to diagnose the subject. For example, test samples of different types collected from the same subject concurrently may indicate the extent of an infection, particularly if the samples are obtained from different locations in the subject or are of different types (e.g. blood and sputum as separate samples) or the extent of the immune response to exposure to an infectious agent or in either case whether the immune response is robust or protective. As another example, test samples of the same type collected from the same subject over time may indicate whether an infection has spread, whether an effective immune response is occurring, whether an immune response is resolving appropriately, or whether a robust or protective immune response has been mounted or is being maintained.

For example, immunoglobulin isotypes exhibit distinct functions, localization, and kinetics during antibody response to an antigen in the body. Thus, in distinguishing between immunoglobulin isotypes, a double-multiplex assay of the present disclosure may provide uniquely comprehensive data as compared to assays that measure total immunoglobulins non-specifically.

A further benefit of some double-multiplex assays as described herein is the ability to quantify the amount of different isotypes of immunoglobulins for different antigens that are present in the test sample, which can provide information regarding the quality and duration of immunity that is not provided by many conventional testing methods.

Although embodiments presented herein often focus on double-multiplex assays as used to detect infectious disease antigens, it will be understood that other antigens may also be detected. For example, antibodies to cancer antigens may be detected to diagnose a cancer, progression or remission of a cancer, details of the immune response to a cancer, or response to treatment of the cancer. As another example, autoantibodies to autoantigens may be detected to diagnose an autoimmune disease, details of the autoimmune response, or response to treatment of the autoimmune disease. Antibodies may similarly be used to detect adverse effects of immune-regulatory therapies, such as autoantibodies formed in cancer patients receiving checkpoint blockade inhibitors. As another example, antibodies directed to allergens, particularly antibodies of the IgE isotype, may be detected to diagnose allergies or response, such as the development of tolerance, to treatments. As yet another example, antibodies directed to organ and tissues for transplant may be detected to determine the suitability of a transplant, development of a rejection-related immune response, potentially before such response leads to actual rejection, or response to anti-rejection treatments, such as development of tolerance. A suitable single biological sources of antigen may be selected in each instance. For example, a virus, bacteria, fungus, parasite, tumor, cancer cell, allergen, autologous tissue, transplanted organ, or vaccine antigen(s) or other vaccine components may be the single biological source. In other assays, it may be beneficial to conduct a single assay to detect antigens from multiple biological sources at once.

The total number of types of data points obtained from the double-multiplex assay may be greater than could be obtained by evaluating some of the different antigen and immunoglobulin isotype combinations via separate ELISAs or LFAs using a test sample of the same size because test sample sizes suitable for double-multiplex assays of the present disclosure may be too small to allow counterpart separate ELISAs or LFAs to be performed for all antigen and immunoglobulin isotype combinations.

The information regarding each type of data point or test sample property may have a predictive value that is at least as good as or better than the predictive value that would be obtained by evaluating each different antigen and immunoglobulin isotype combination via separate ELISAs or LFAs.

Another potential benefit of a double-multiplex assay as described herein is that using multiple types of data points to determine a test sample property may increase assay specificity without a corresponding sacrifice of sensitivity. Specificity is a measure of the number of positive test samples that are correctly identified. Assessing more types of data points increases the probability that the assay will correctly identify true positives, thereby enhancing specificity. Sensitivity is a measure of the number of negative test samples that are correctly identified. Typically, the ability of an assay to identify the maximum number of true positive results comes at the cost of an increased number of false negative results. In other words, an increase in specificity often results in a decrease in sensitivity. In the methods disclosed herein, however, the positivity threshold for each of the multiple types of data points is determined individually. Thus, unlike many conventional assays that do not operate in a multiplex fashion, the double-multiplex assays described herein provide both excellent sensitivity and excellent specificity.

Some double-multiplex assays of the present disclosure may also reduce time or cost to determine a test sample property as compared to conventional methods by conducting a single assay, rather than multiple assays, to evaluate the presence of various immunoglobulin isotypes or antibodies against multiple etiologic pathogens.

Furthermore, the ability to use small-volume test samples in some double-multiplex assays of the present disclosure may facilitate more frequent and less invasive sample collection as compared to conventional assays. The use of small-volume test samples, and, in particular, sub-microliter test samples also facilitates adaptation of the assays to direct-to-consumer applications and sample collection in non-medical settings.

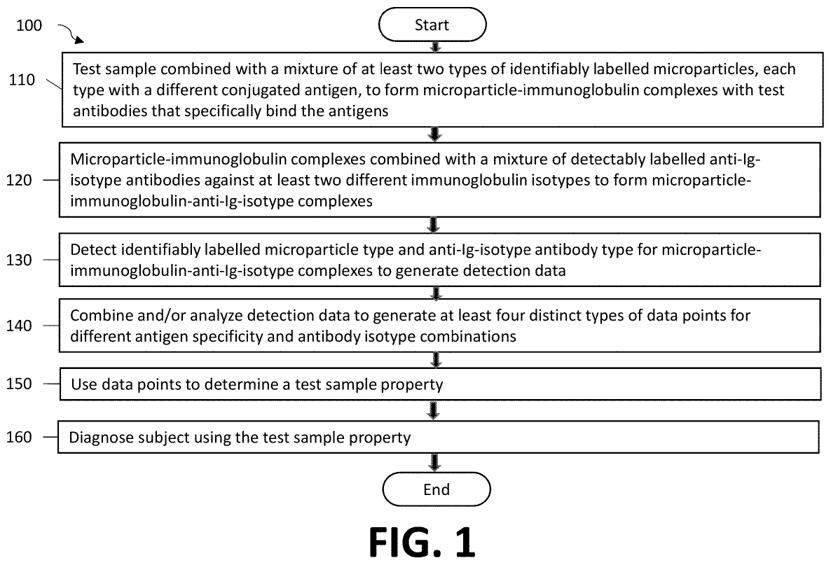

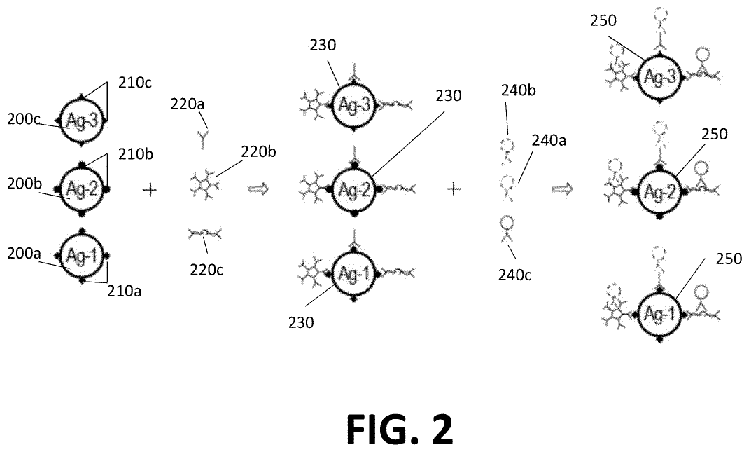

Referring now to the embodiment presented in FIGS. 1-2, which may be combined with all other aspects of the disclosure, FIG. 1 provides a flow chart of a double-multiplex assay 100 according to the present disclosure. FIG. 2 provides schematic diagrams of compositions used in or created by the double-multiplex assay of FIG. 1. Although the embodiment of FIGS. 1-2 uses three antigens and detects three immunoglobulin isotypes to obtain nine types of data points, the embodiment may be readily adapted using the teachings of the present disclosure to use as few as two antigens to detect as few as two immunoglobulin isotypes to obtain four types of data points, or to detect more different antigens or immunoglobulin isotypes to obtain more types of data points.

As used herein, the term “antigen” refers to a protein polypeptide, peptide, DNA, RNA, polynucleic acid, nucleic acid, or allergen that is capable of triggering an immune response in a subject. An antigen may be associated with a disease-causing agent, such as a bacterium, a virus, or a fungus, or it may be a protein or peptide that is capable of triggering an allergic or an autoimmune reaction in a subject.

As used herein, the terms “antibody” and “immunoglobulin” are interchangeable, and refer to the immunological proteins that are developed within a host subject's body or by tissue culture methods to have an affinity for a target antigen. An antibody or immunoglobulin is said to be “against” or to “bind” an antigen to which it has affinity. Immunoglobulins (Ig) occur in multiple isotypes, including IgG, IgM, IgA, IgE, and IgD. Certain isotypes are further divided into sub-types. For example, the IgG isotype comprises the subtypes IgG1, IgG2, IgG3, and IgG4. As used herein, the term “isotype” includes both isotypes and sub-types of isotypes.

As used herein, the term “epitope” refers to the portion of any antigen to which an antibody binds. One antigen may include multiple epitopes and different antibodies against the same antigen may bind to the same or different epitopes of that antigen. Although the discussion herein focuses on double-multiplex assays using different antigens, similar assays may also be conducted using two or more different epitopes of the same antigen when it is useful to obtain types of data points that are specific to epitopes rather than entire antigens.

As used herein, the term “test sample” refers to a sample which is to be assayed for the presence of immunoglobulins that bind the target antigen(s). The test sample is a biological sample from a subject. Examples of test samples include, but are not limited to, whole blood, serum, plasma, nasal secretions, sputum, bronchial lavage, urine, stool, saliva, sweat, and cells that have membrane immunoglobulin (such as memory B cells).

As used herein, term “about” means±20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, an “assay” may sometimes also be referred to as a “test.”

The double-multiplex assay 100 of FIG. 1 detects test antibodies in a test sample. In step 110, a test sample from a subject is combined with at least two types of identifiably labelled microparticles, each with a different conjugated antigen, under conditions that allow test antibodies in the test sample to specifically bind any antigen on an identifiably labelled microparticle to which the test antibody has affinity to form microparticle-immunoglobulin complexes.

In some embodiments, the identifiably labelled microparticles are combined with the test sample for a period of time to facilitate formation of the microparticle-immunoglobulin complexes. For example, the period of time of step 110 may be 1 minute, 2 minutes, 5 minutes, 10 minutes, 20 minutes, or an interval between any of these times.

In some embodiments, the test sample is whole blood, serum, plasma, interstitial fluid, nasal secretions, sputum, bronchial lavage, urine, stool, saliva, or sweat from a subject. In certain embodiments, the test sample is whole blood, serum, or plasma. The test sample may have a volume of 0.1 μl or more, such as a volume of 0.1-0.5 μl, 0.1-0.7 μl, 0.1-0.9 μl, 0.1-2.0 μL, 0.1-3.0 μL. 0.1-5.0 μL, 0.1-10.0 μL, 0.1-15.0 μL, or 0.1-20.0 μL. In some embodiments, the biological sample volume is 0.1 μl, 0.2 μl, 0.3 μl, 0.4 μl, 0.5 μl, 0.6 μl, 0.7 μl, 0.8 μl, 0.9 μl, 1.0 μl, 1.1 μl, 1.2 μl, 1.3 μl, 1.4 μl, 1.5 μl, 1.6 μl, 1.7 μl, 1.8 μl, 1.9 μl, 2.0 μl, 2.1 μl, 2.2 μl, 2.3 μl, 2.4 μl, 2.5 μl, 2.6 μl, 2.7 μl, 2.8 μl, 2.9 μl, 3.0 μl, 3.1 μl, 3.2 μl, 3.3 μl, 3.4 μl, 3.5 μl, 3.6 μl, 3.7 μl, 3.8 μl, 3.9 μl, 4.0 μl, 4.1 μl, 4.2 μl, 4.3 μl, 4.4 μl, 4.5 μl, 4.6 μl, 4.7 μl, 4.8 μl, 4.9 μl, 5.0 μl, 5.5 μl, 10 μl, 10.5 μl, 11 μl, 11.5 μl, 12 μl, 12.5 μl, 13 μl, 13.5 μl, 14 μl, 14.5 μl, 15 μl, 15.5 μl, 16 μl, 16.5 μl, 17 μl, 17.5 μl, 18 μl, 18.5 μl, 19 μl, 19.5 μl, or 20 μl. The test sample may be used unaltered or components, such a stabilizing agent found in a collection vial, may be mixed with the test sample during the collection process. In instances where components are mixed with the test sample during the collection process, the test sample volume is the volume actually obtained from the subject, not the volume after mixing with components during the collection process. In such instances, the test sample volume may be estimated by subtracting any volume estimated to be contributed by components mixed with the sample during the collection process from the volume present after such mixing.

In some embodiments, the test sample is diluted before being assayed. For example, the test sample may be diluted 1:40, 1:30, 1:20, 1:10, 1:5, 1:2, or 1:1. Appropriate buffers for sample dilution are well known in the art. In some embodiments, the test sample is diluted in PBS buffer containing 1% bovine serum albumin (BSA). The test sample volume does not include any diluent volumes.

Test samples may be used in step 110 of the double-multiplex assay immediately, within about 5 minutes, within about 10 minutes within about 30 minutes, within about 60 minutes, within about 2 hours, within about 12 hours, within about 24 hours, within about 48 hours, or during a time interval between about any of these time points after collection of the test sample from the subject. Appropriate stabilization or preservative components may be added to the test sample, particularly if longer periods of time will elapse between collection and use in step 110 of the double-multiplex assay. Test samples may be frozen if needed.

Test samples may also result from processing of a sample as directly obtained from a patient. For example, if the test sample is plasma, it may be obtained by centrifuging a whole blood sample as directly obtained from a patient.

Test samples may be collected using any suitable methods and containers. For example, whole blood, serum, or plasma may be collected by venipuncture in a vacuum tube. Whole blood, serum, or plasma may also be collected by finger stick and a capillary action device. Whole blood, serum, plasma, or interstitial fluid may be collected using an alternative site stick, such as an arm stick as is commonly used in glucose monitoring, and a capillary action device. Samples secreted or expelled by the subject may simply be collected using standard laboratory processes and equipment. Bronchoalveolar lavage samples may be collected using a bronchoscope. In the limited instance of brochoalveolar lavage, the test sample volume may include the fluid introduced into the airway in order to obtain the test sample.

The test sample may be diluted prior to combining with the identifiably labelled microparticles. In some embodiments, it may be diluted to a volume of 20-50 μl.

The microparticles used in step 110 may include microparticles 200 illustrated in FIG. 2. The microparticles 200 may be of any appropriate size and shape for use in the double-multiplex assay 100 and may have micrometer- or nanometer-scale cross-section dimensions. Microparticles may also be referred to as beads. In certain embodiments, the microparticles 200 have a cross-section that is from 0.001 μm to 1000 μm in length, 0.01 μm to 100 μm in length, 0.1 μm to 50 μm in length, 0.1 μm to 10 μm in length, 1 μm to 10 μm in length, 1 μm to 6 μm in length, 1 μm to 5 μm in length, or 1 μm to 3 μm in length. In certain embodiments, the microparticles are spherical or approximately spherical, in which case the cross-section may be a diametric cross-section and the microparticles may be referred to as microspheres. Microparticles have a surface to which molecules may be attached. Such attached molecules are referred to as being conjugated to the microparticle.

As used herein, the term “identifiably labelled” refers to microparticles or molecules having chemical or physical characteristics that permit different types of microparticles or molecules to be distinguished. For example, each identifiably labelled microparticle of a given type can be distinguished from identifiably labelled microparticles of a different type. Any appropriate identifiable label may be used, including size, magnetic properties, fluorescence properties (such as excitation or emission wavelength or intensity, for example using ultraviolet excitation or violet excitation) and metal isotope properties. The identifiable label may be a property of the microparticle or molecule itself, or it may result from conjugation of a label to the microparticle or molecule. Each different type of microparticle having a different antigen bound to it has a different and distinct identifiable label.

In the embodiment illustrated in FIG. 2, three types of identifiably labelled microparticles are illustrated, type 200a, type 200b, and type 200c. Identifiably labelled microparticle type 200a has a different identifiable label than identifiably labelled microparticle type 200b and identifiably labelled microparticle type 200c. Identifiably labelled microparticles types 200b and 200c similarly have different and distinct identifiable labels.

Each type of identifiably labelled microparticle may have a surface upon which an antigen 210 is attached. In some embodiments, each type of identifiably labelled microparticle may have a different antigen attached. For example, the different types of identifiably labelled microparticles 200a, 200b, and 200c in FIG. 2 each have a different type of antigen 210a, 210b, and 210c, respectively, attached. In some embodiments, such as that illustrated in FIG. 2, each type of identifiably labelled microparticle has only one distinct antigen attached.

An antigen 210 may be conjugated to the surface of an identifiably labelled microparticle 200 directly or via a peptide or polypetide attached to the surface. The antigen 210 may be conjugated to the surface by any type of binding interaction including ionic bonding, hydrogen bonding, covalent bonding, Van der Waals, and hydrophilic/hydrophobic interactions. Each identifiably labelled microparticle may be conjugated to multiple copies of its antigen. Type of antigens 210 that may be conjugated to microparticles 200 include polypeptides, proteins, and nucleic acids.

In some embodiments, the different and distinct label for an identifiably labelled microparticle 200 may be conjugated to the microparticle by being attached to the antigen 210 either prior to or after conjugation of the antigen 210 to the microparticle 200.

At least two types of identifiably labelled microparticles 200 with at least two different antigens are combined with the test sample in double-multiplex assay step 110. In some embodiments, three, between two and four, between two and five, between two and six, between two and seven, between two and eight, between two and nine, between two and ten, between two and twenty, between two and fifty, between two and one hundred, of between two and five hundred types of identifiably labelled microparticles 200 are combined with the test sample in double-multiplex assay step 110. In some embodiments between two and four, between two and five, between two and six, between two and seven, between two and eight, between two and nine, between two and ten, between two and twenty, between two and fifty, between two and one hundred, of between two and five hundred different antigens 210 are conjugated to identifiably labelled microparticles 200 that are combined with the test sample in double-multiplex assay step 110.

During double-multiplex assay step 110, test antibodies 220 in the test sample that are against an antigen on a identifiably labelled microparticle specifically bind to that antigen to form microparticle-immunoglobulin complexes 230.

Test antibodies 220 in the test sample may be of only one isotype, or multiple isotypes. In the embodiment illustrated in FIG. 2, test antibodies 220 include IgGs 220a, IgMs 220b, and IgAs 220c. Other possible isotypes, not illustrated, include IgEs and IgDs. Microparticle immunoglobulin complexes 230 all contain three isotypes of test antibodies 220 bound to the respective antigens 210. However, microparticle immunoglobulin complexes 230 may contain only one isotype of a test antibody 220 if the test sample does not contain other isotypes. For example, early in the immune response of a subject to an infectious agent containing the antigen, the test sample may only contain the IgM isotype because this isotype can be expressed by B cells without isotype switching.

Depending on the antigens 210, it may be possible that one type of identifiably labelled microparticle may form a microparticle-immunoglobulin complex 230 containing only one antibody isotype, while a different type of identifiably labelled microparticle with a different antigen may form a microparticle-immunoglobulin complex containing additional antibody isotypes. Such a situation may result, for example, if the antigen on the first type of identifiably labelled microparticle is unique to an infectious agent the subject was only recently exposed to and, therefore, has only produced IgMs against, while the antigen on the second type of identifiably labelled microparticle is common to both the recent infectious agent and another infectious agent to which the subject was exposed a longer time in the past, allowing B cell isotype switching.

Typically, each of the identifiably labelled microparticles 200 contains sufficient copies of the antigen 210 to allow all isotypes of test antibodies 220 against the antigen 210 found in the test sample to also be present in the majority of the microparticle-immunoglobulin complexes 230 formed.

Upon completion of step 110, in some embodiments of the double-multiplex assay the microparticle-immunoglobulin complexes are washed under conditions that do not substantially disrupt the complex. For example, the microparticle-immunoglobulin complexes may be washed with phosphate-buffered saline (PBS). This may remove unbound test sample components from the microparticle-immunoglobulin complexes, which may then be placed in an appropriate liquid to maintain the complexes, such as additional PBS.

In other embodiments of the double-multiplex assay 100, the double-multiplex assay proceeds directly from step 110 to step 120 without washing.

In step 120, the microparticle-immunoglobulin complexes are combined with anti-Ig-isotype antibodies against two different Ig isotypes under conditions that allow the anti Ig-isotype antibodies to specifically bind test antibodies in microparticle-immunoglobulin complexes to which the anti-Ig-isotype antibody has affinity to form sufficient to allow to form microparticle-immunoglobulin-anti-Ig-isotype complexes.

The anti-Ig-isotype antibodies may be combined with the microparticle-immunoglobulin complexes as a mixture of antibodies in a single step, as multiple mixtures in multiple sequential steps, or one-at-a-time in sequential steps. For example, the microparticle-immunoglobulin complexes may be first combined with anti-IgG antibodies, then combined with anti-IgM antibodies, then anti-IgA antibodies, and so forth, until all desired anti-Ig-isotype antibodies have been combined with the microparticle-immunoglobulin complexes. In the case of sequential steps, in some embodiments the microparticle-immunoglobulin complexes may be washed between steps.

In some embodiments, the microparticle-immunoglobulin complexes are combined with the anti-Ig-isotype antibodies, either as a mixture or in each step if sequential steps are used, for a period of time to facilitate formation of the microparticle-immunoglobulin-anti-Ig-isotype complexes. For example, the period of time of step 120 may be 1 minute, 2 minutes, 5 minutes, 10 minutes, 20 minutes, or an interval between any of these times.

In the embodiment depicted in FIG. 2, three different types of anti-Ig-isotype antibodies 240 are provided. Anti-Ig antibody 240a specifically binds IgM antibodies. Anti-Ig antibody 240b specifically binds IgG antibodies. Anti-Ig antibody 240c specifically bind IgA antibodies. However, the anti-Ig-isotype antibodies 240 of step 120 may be against at least two, at least three, at least four, at least five, at least six, at least seven, or at least eight immunoglobulin isotypes or subtypes.

Example microparticle-immunoglobulin-anti-Ig-isotype complexes 250 are also depicted in FIG. 2. In these examples, for each identifiably labelled microparticle 200 used in step 110, a microparticle-immunoglobulin-anti-Ig-isotype complex 250 also containing three isotypes of test antibody 220 and three different anti-Ig antibodies 240c is formed. However, depending on the antigens 210, it may be possible that one type of identifiably labelled microparticle may form a microparticle-immunoglobulin-anti-Ig-isotype complex 250 containing only one test antibody isotype and, as a result, only one type of anti-Ig antibody, while a different type of identifiably labelled microparticle with a different antigen may form a microparticle-immunoglobulin-anti-Ig-isotype complex containing additional test antibody isotypes and, as a result, additional anti-Ig antibodies.

Typically, each of the identifiably labelled microparticles 200 contains sufficient copies of the antigen 210 to allow all isotypes of test antibodies 220 against the antigen 210 found in the test sample and specifically bound anti-Ig-isotype antibodies to also be present in the majority of the microparticle-immunoglobulin-anti-Ig-isotype complexes 250 formed. The anti-Ig-isotype antibodies 240 are detectably labelled prior to use in step 120 As used herein, the term “detectably labelled” refers to particles or molecules having chemical or physical characteristics that permit the presence or quantity of the particles or molecules to be detected. Detectable labels include, but are not limited to, fluorescence properties, luminescent properties, and colorimetric properties. A distinguishable label may be, for example, a specific fluorescence intensity, frequency, or combination of frequencies. Examples of labels having fluorescent properties are green fluorescent protein, fluorescein, and phycoerythrin. Each different type of anti-Ig-isotype antibody has a different and distinct detectable label, allowing the antibodies to be distinguished.

In the embodiment illustrated in FIG. 2, three types of detectably labelled anti-Ig-isotype antibodies 240 are illustrated, type 240a, type 240b, and type 240c. Detectably labelled anti-Ig-isotype antibody type 240a has a different detectable label than detectably labelled anti-Ig-isotype antibody type 240b and anti-Ig-isotype antibody type 240c. Detectably labelled anti-Ig-isotype antibodies types 200b and 200c similarly have different and distinct identifiable labels.

Upon completion of step 120, in some embodiments the microparticle-immunoglobulin-anti-Ig-isotype complexes are washed under conditions that do not substantially disrupt the complex. For example, the microparticle-immunoglobulin-anti-Ig-isotype complexes may be washed with phosphate-buffered saline (PBS). This may remove unbound anti-Ig-isotype antibodies from the microparticle-immunoglobulin-anti-Ig-isotype complexes, which may then be placed in an appropriate liquid to maintain the complexes or to allow detection in step 130, such as additional PBS.

In other embodiments of the double-multiplex assay 100, the double-multiplex assay proceeds directly from step 120 to step 130 without washing.

In step 130, the microparticle-immunoglobulin-anti-Ig-isotype complexes are placed in a detector that detects, for individual microparticle-immunoglobulin-anti-Ig-isotype complexes, the microparticle type by detecting the identifiable label and anti-Ig-isotype by detecting the detectable label to generate detection data. The identity of the identifiably labelled microparticle in each detected microparticle-immunoglobulin-anti-Ig-isotype complex as well as the presence or absence of or, more typically, the amount of anti-Ig-isotype antibody against each isotype assayed may be collected or stored separately for each complex, or collected or stored in aggregate based on identifiably labelled microparticle type. Alternatively or in addition, the identity of the anti-Ig-isotype antibody in each detected microparticle-immunoglobulin-anti-Ig-isotype complex as well as the presence or absence of or, more typically, the number of each type of identifiably labelled microparticle used in the double-multiplex assay may be collected or stored separately for each complex, or collected or stored in aggregate based on anti-Ig-isotype antibody type. Collection and storage in this context involves the use of a processor or memory in communication with or part of the detector.

In certain embodiments, the microparticle-immunoglobulin-anti-Ig-isotype complexes are sorted or counted. In some embodiments, the detector is a flow cytometer. For example, each type of identifiably labelled microparticle may be distinguished based on its distinguishing properties, and the anti-Ig-isotype antibody or antibodies in a complex with a given type of identifiably labelled microparticle may be identified based on their detectable labels. In some embodiments, the microparticles are identifiably labelled by fluorescence properties and the anti-Ig-isotype antibodies are fluorescently labelled, and the analysis is carried out using multi-color flow cytometry. In some embodiments, the microparticles are identifiably labelled by ultraviolet-excited or violet-excited fluorescence properties, the anti-Ig-isotype antibodies are fluorescently labelled, and the analysis is carried out using multi-color flow cytometry.

In some embodiments, the microparticles are identifiably labelled by metal isotope and the anti-Ig-isotype antibodies are metal isotope labelled, and the detector is a multi-metal isotope mass cytometer.

In some embodiments, the detector uses a mass cytometry method, such as CyTOF® (Fluidigm, Calif.). CyTOF®, also known as cytometry by time of flight, is a technique based on inductively coupled plasma mass spectrometry and time of flight mass spectrometry. In this technique, isotopically pure elements, such as heavy metals, are conjugated to antibodies. The unique mass signatures are then analyzed by a time of flight mass spectrometer.

As used herein, the term “control” refers to a reference standard. A positive control is known to provide a positive test result. A negative control is known to provide a negative test result. Positive and negative control samples, microparticles, and anti-Ig-isotype antibodies may also be included in the double-multiplex assay and detected as appropriate in step 130 or in a separate double-multiplex assay to provide additional detection data.

In step 140, the detection data is combined or analyzed to generate at least four distinct types of data points for different antigens and antibody isotypes. The combination or analysis may be performed by an appropriately programmed processor provided with the detection data. The data points may be stored in memory associated with the processor.

The combination of different identifiably labelled microparticles for different antigens and detectably labelled anti-Ig-isotype antibodies allows the detection not only of test antibodies present in the test sample that bind the target antigen(s), but also of the isotype or subtype of those test antibodies present. Further, the presently disclosed methods not only detect the presence of immunoglobulins, but provide quantitative or semi-quantitative data regarding the levels of each isotype or subtype of immunoglobulin that binds to each of the test antigens as separate date points.

Each possible combination of antigen and immunoglobulin isotype yields a distinct type of data point. In its simplest form, the double-multiplex assay detects test antibodies against at least two different antigens and it simultaneously also detects at least two different immunoglobulin isotypes of the test antibodies. Such a double-multiplex assay provides a total of four types of data points regarding test antibodies present in the test sample. In an exemplary more complex variation, such as a double-multiplex assay using the materials of FIG. 2, the assay may detect test antibodies against three different antigens while simultaneously detecting at least three different immunoglobulin isotypes of the antibodies. Such a double-multiplex assay provides a total of nine types of data points regarding test antibodies present in the test sample.

In general, the number of types of data points obtainable=number of different antigens on identifiably microparticles x number of different immunoglobulin isotypes detected. The maximum number of types of data points is limited primarily by detection capabilities of the detector and may be quite high, such as 50, 100, or 1000. Although the double-multiplex assay can generate a microparticle-immunoglobulin-anti-Ig-isotype complex that corresponds to each obtainable data point, the assay does not necessarily have to detect each microparticle-immunoglobulin-anti-Ig-isotype complex in step 130 or provide a data point for each such complex in step 140. For example, in some instances, certain antigen-antibody isotype combinations may simply not be of value and may be undetected or not used to generate data points. This may improve accuracy of types of data points of interest or allow quicker assay results.

In some embodiments steps 130 and 140 may be performed simultaneously, nearly simultaneously, or in an unparseable fashion by the detector or the detector and a processor and memory in communication with the detector.

In some specific embodiments, in step 130 or combined steps 130 and 140, each type of identifiably labelled microparticle is distinguished and gated by its unique characteristics (e.g. size or intensity of fluorescence or heavy metal isotopes) and the fluorescence intensities (FI) of multiple fluorochromes or the heavy metal intensities (HMI) of the microparticle-immunoglobulin-anti-Ig-isotype complexes are measured and proportionally correlated with the concentrations of corresponding isotypes of test antibodies against the same antigen.

The period of time for steps 110 through 140 may be about 5 minutes, about 10 minutes about 30 minutes, about 60 minutes, about 2 hours, about 3 hours, about 6 hours, about 12 hours, about 24 hours, about 48 hours, or during a time interval between about any of these time points. In a specific embodiment, the period of time for steps 110 through 140 may be between 1 hour and 2 hours or between 30 minutes and 3 hours.

Next, in step 150, the data points are used to determine a test sample property. The test sample property may be determined by subjecting the data points to further mathematical analysis, such as comparison to a threshold to determine positive of negative status.

For instance, if the test sample is from a subject who may have been exposed to an infectious disease, then the data points may be subjected to further mathematical analysis to determine whether the data points are consistent with the subject having actually been exposed to the disease. Other related test sample properties include whether the subject has mounted a robust immune response to the disease or whether the subject has mounted a protective immune response to the disease.

As another example, the test sample property may be whether the subject contains autoantibodies or whether the autoantibodies are present in amounts and types that likely correlated with a harmful autoimmune response.

Other test sample properties described herein may also be determined.

With respect to double-multiplex assay accuracy for a test sample property, several metrics may be used herein as descriptors, including “sensitivity”, “specificity”, “concordance”, “positive predictive value”, “negative predictive value,” “false positive rate,” and “false negative rate.” These metrics for a simple positive or negative test sample property determined using a given assay can be defined by the following formulas as a function of the number of “True Positive” (TP), “True Negative” (TN), “False Positive” (FP) and “False Negative” (FN) cases:

Sensitivity=TP/(TP+FN);

Specificity=TN/(TN+FP);

Concordance (Correlation)=(TP+TN)/(TP+TN+FP+FN),

Positive Predictive Value=TP/(TP+FP);

Negative Predictive Value=FN/(FN+TN);

False Positive Rate=FP/(TP+FP); and

False Negative Rate=FN/(TN+FN).

As used herein, “predictive value” encompasses both positive predictive value and negative predictive value.

The number and type of data points obtainable in the double-multiplex assay or used in further mathematical analysis may be selected so that the test sample property may be determined with at least a minimum accuracy. In some embodiments, the test sample may be smaller than would be required to obtain the same number of types of data points using a non-multiplexed ELISA or LFA and the same type of test sample, antigens, and immunoglobulin isotype detection methods at all or with the ability to provide the same accuracy in determining the test sample property.

In another example, the number and type of data points may be selected so that the double-multiplex assay has at least a minimum predictive value. In some embodiments, this minimum accuracy may be at least as high as what would be provided by a non-multiplexed ELISA or LFA using the same type of test sample, antigens, and immunoglobulin isotype detection methods. In some examples where the test sample property is a simple positive or negative, a double-multiplex assay of the present disclosure may have a specificity 10 times or 100 times higher than a corresponding set of ELISAs or LFAs or other corresponding assays in which a single type of data point is used to determine the test sample property. In general, the specificity of the test sample property is increased without a decrease in sensitivity as compared to a corresponding assay that uses only a single type of data point to determine the test sample property.

More complex test sample properties may also be determined using the data points, such as ratios of sample antibodies against different antigens, ratios of isotypes of sample antibodies, and more complex properties such as ratios of combined data points.

A sample test property may be determined using all of the data points generated in step 140, or one or more sets of fewer than all of the data points. For example, typically a test sample property corresponding to a given antigen will be determined using only the set of data points generated from microparticle-immunoglobulin-anti-Ig-isotype complexes containing that antigen. As another example, a test sample property may be determined using only data points that meet a given threshold for an indicator of accuracy.

Although in some embodiments, only a single test sample property is determined in step 150, in other embodiments two or more test sample properties are determined in step 150. If two or more test sample properties are determined in step 150, they may be determined using the same data points in some embodiments or different sets of data points in other embodiments.

For example, the same set of data points may be used to determine if the test sample is positive or negative for antibodies against a specific antigen and also, as a separate test sample property, the relative amounts of antibody isotypes against the specific antigen, the prevalent antibody isotype against the specific antigen, an estimated amount of time since the subject providing the test sample was first exposed to the antigen, or whether the subject providing the test sample is likely to amount an effective immune response if re-exposed to the antigen.

In another example, data points for IgG, IgA, and IgM immunoglobulin isotypes against a given antigen may all be used to determine if the test sample is positive or negative for antibodies against the antigen, but, in some embodiments, only data points for IgA and IgG may be used to determine whether the subject providing the test sample is likely to amount an effective immune response if re-exposed to the antigen.

In another example, date points for multiple antibody isotypes against a given allergen, may be used to determine if the subject has been exposed to the allergen, but only IgE or a combination of IgE and other specific isotypes may be used to determine if the patient is likely to have a harmful allergic response to the allergen.

Additionally, test sample properties may be determined by relying on other test sample properties. For example, data points corresponding to different antibody isotypes all against the same antigen may be used to determine a test sample property of positive or negative status for that antigen by correlating the data points. Positive and negative status for each antigen in the double-multiplex assay may be determined as separate test sample properties, then those test sample properties may be used to determine a final test sample property of whether the subject has antibodies against a common source of all the antigens, such as a virus or tumor that expresses all of the antigens.

Various indicators of accuracy may also calculated for positive and negative status at each iteration of this process and used to generate final accuracy data for the ultimate positive or negative exposure determination. Indicators of accuracy calculated are concordant results, discordant results, relative sensitivity, relative specificity, concordance, positive predictive value, negative predictive value, false positive rate (100%-positive predictive value), and false negative rate (100%-negative predictive value). Indicators of accuracy may be used in more complex mathematical analysis, such as weighting of data points or types of data points in calculations. They may also be used to exclude certain data points that do not meet accuracy thresholds from any test sample property determination. Finally, indicators of accuracy may be further processed to arrive at indicators of accuracy for test sample properties that are calculated from data points or other test sample properties.

Correlation of the data points may involve any of a number of types of mathematical analysis which may take into account the raw data for the data point, a simple positive or negative indictor for that data point, and one or more indicators of accuracy.

In one embodiment, data points may reflect immunoglobulin isotypes against a first antigen. The test sample may be deemed to be positive or negative for the first antigen based on concordance of the data points. For example, if three immunoglobulin isotypes were assayed, the test sample may be deemed positive or negative for the first antigen based on simple concordance of positive or negative status for each immunoglobulin isotype. So, if data points for two of the immunoglobulin isotypes are negative, then the test sample is deemed negative for antibodies against the first antigen.

More complex analysis may also be conducted where, for example, results for one immunoglobulin isotype are weighted more heavily than for another immunoglobulin isotype. Weighting may be pre-set or it may be adjusted to reflect the relative accuracy of the data point for each isotype. Such weighting may be particularly useful in concordance determinations where an even number of data points or types of data points.

Using the sample example embodiment, the test sample may be deemed positive or negative for exposure to a source of multiple antigens assayed. For example, if a second antigen is present, then positive or negative status may be determined for that antigen. The test sample may then be deemed overall positive or negative for exposure to the source of both antigens if it is positive for antibodies against either antigen. In another variation, a third antigen may be assayed and the sample may be deemed overall positive or negative for exposure to the source of all three antigens based on concordance of the antigen-specific results. More complex analysis, similar to those described above with respect to immunoglobulin isotype-specific results for a single antigen, may also be used.

Determining a test sample property may be performed by an appropriately programmed processor provided with the detection data or data points. The test sample property may be stored in memory associated with the processor.

In step 160, which may be omitted in some embodiments, the subject is diagnosed using at least one test sample property determined in step 150. For instance, the subject may be diagnosed with having an infectious disease, having been exposed to an infectious disease, having mounted a robust immune response to the infectious disease, or having developed a protective immune response to the infectious disease. Other diagnoses described herein may also be made using the test sample properties.

In another example, the diagnosis may involve analysis of multiple test samples taken from the subject at the same time or at different times. Such analysis may involve further mathematical analysis using an appropriately programmed processor. For example, the diagnosis may involve determining the duration of test antibodies against an antigen in the patient or determining the isotype or amount of test antibodies against an antigen over time in the patient.

In some embodiments the present disclosure provides kits to simultaneously detect multiple immunoglobulin isotypes against multiple different antigens so as to provide distinct types of data points for different antigen and immunoglobulin isotype combinations. Such kits may contain materials as described above in the context of a double-multiplex assay and, more specifically, as shown in FIG. 2. In some embodiments, the kits include at least two types of identifiably labelled microparticles. Each type of identifiably labelled microparticles may, in some embodiments, be conjugated to a different antigen. The different antigens may be included in the kit or provided by the user. Reagents for such conjugation may be provided in the kit. In other embodiments, each type of identifiably labelled microparticle is conjugated to a different antigen. The kit also includes at least two anti-Ig-isotype antibodies against at least two immunoglobulin isotypes. In some embodiments, each type of anti-Ig-isotype antibody has a different detectable label. On other embodiments, each type of anti-Ig-isotype antibody may be conjugated to a detectable label. The detectable labels may be included in the kit or provided by the user. Reagents for such conjugation may be provided in the kit.

In some embodiments, kits may further comprise positive or negative control samples, finger stick needles or blades, sample collection containers, supplies for returning a sample for analysis, such as a mailing kit or container appropriate for transport by courier, instructions for use, or any combination thereof.

In a specific embodiment, the double-multiplex assay detects exposure of a subject SARS-CoV-2, development of a robust immune response to SARS-CoV-2, or development of a protective immune response to SARS-CoV-2. Aspects of this embodiment not specifically discussed here may be in any manner described in the present specification. In the SARS-CoV-2 assay, the antigens include at least two antigens of SARS-CoV-2. More specifically the antigens are S1, RBD, and NP. The identifiably labelled microparticles are fluorescently-labelled microspheres. The anti-Ig-isotype antibodies are anti-IgG, anti-IgM, and anti-IgA and are fluorescently-labelled. Detection uses a flow cytometer able to detect fluorescence of all fluorescent labels on the microspheres and anti-Ig-antibodies. Fluorescence data acquired during detection is separately gated for the unique fluorescence signature of each identifiably labelled microsphere, thereby restricting the data to that associated with a single type of identifiably labelled microsphere and, hence, a single antigen and test antibodies against that antigen. Within the gated data set corresponding to each type of identifiably labelled microsphere, fluorescence intensity associated with each type of anti-Ig-isotype antibody complex is identified and used to generate a data point associated with the specific type of identifiably labelled microsphere and anti-Ig-isotype and hence, the specific antigen and immunoglobulin isotype. The data point is then compared to a threshold for that type of data point and the test sample is deemed positive or negative for a test antibody against the specific antigen having the specific immunoglobulin type.

The data point is then correlated with data points for the same antibody isotype against the three antigens and the test sample is deemed to be positive or negative with respect to antibodies of that isotype against SARS-CoV-2 based on concordance of the results. For example, if the test sample, if the data point for S1 IgG was negative, the data point for RBD IgG was negative, and the data point for NP IgG was positive, the test sample would be deemed negative for IgG antibodies against SARS-CoV-2 due to concordance.

The positive or negative status of the test sample for test antibodies of the three isotypes antigens is then correlated with an overall positive or negative status of the test sample with respect to prior exposure of the subject to SARS-CoV-2. For example, if the test sample is positive for any of the three antibody isotypes against SARS-CoV-2, then the test sample may be designated as overall positive for SARS-CoV-2 antibodies, indicative of exposure of the patient to SARS-CoV-2.

Positive or negative status for a robust immune response to SARS-CoV-2 or a protective immune response against SARS-CoV-2 may be determined in a similar manner, but with higher required threshold amounts of antibody levels or greater requirements for IgG and IgA antibodies as opposed to simply IgM antibodies.

Antibody levels in this SARS-CoV-2 assay may be compared using at least two assays on samples obtained at different times to determine if the subject is developing a more mature or robust immune response, typically due to decreases of overall IgM antibody levels, increases in overall IgG or IgA levels or IgG or IgA levels relative to IgM levels, or development of antibodies against additional antigens.

Sensitivity of this assay is enhanced as compared to traditional ELISAs because, while levels of one immunoglobulin isotype may be low for one antigen, levels of that isotype may be higher for the other two antigens, reducing the chances of false negatives.

Various indicators of accuracy are also calculated for positive and negative status at each iteration of this process and used to generate final accuracy data for the ultimate positive or negative exposure determination. Indicators of accuracy calculated are concordant results, discordant results, relative sensitivity, relative specificity, concordance, positive predictive value, negative predictive value, false positive rate, and false negative rate.

In a related specific embodiment, a kit for detecting exposure to SARS-CoV-2 is provided that includes a first type of microsphere labelled with a first distinct fluorescent label and conjugated to SARS-CoV-2 S1 antigen, a second type of microsphere labelled with a second distinct fluorescent label and conjugated to SARS-CoV-2 RBD antigen, and a third type of microsphere labelled with a third distinct fluorescent label. The kit also includes anti-IgG antibodies with a fourth distinct fluorescent label, anti-IgA antibodies with a fifth distinct fluorescent label, and anti-IgM antibodies with a sixth distinct fluorescent label. The kit may be used with a test sample to generate nine types of data points related to positive of negative status for each antibody isotype for each antigen, which indicate whether the subject who provided the test sample has been exposed to SARS-CoV-2.

The kit may further include washes, buffers, and sample collection implements.

In a specific embodiment, the kit may include:
Instructions for use, including instructions for control preparation
   Reagents to perform 100 tests
   Reagent #1: SARS-CoV-2 antigen coated microspheres
   Reagent #2: Sample dilution buffer
   Reagent #3: Fluorescent tagged secondary antibodies Components required but not provided in the kit include:
Flow cytometer
Standard wash buffer
Standard flow cytometry suspension buffer
Microtiter plates
Pipettes
Positive and negative control samples

### EXAMPLES

### Example 1

### Simultaneous Detection of Multiple Immunoglobulin Isotypes Against a Single Antigen

Single antigen-conjugated microspheres with a fluorescent signature, in which the antigen was RBD, were incubated with a test sample, allowing the immunoglobulins present in the sample to bind to the antigen on the surface of the microspheres. Test samples were plasma samples from SARS-CoV-2 patients (n=5, positive status confirmed by RT-PCR) or negative control patients n=5, samples collected 2 years prior to emergence of SARS-CoV-2).

After washes, the microspheres were sequentially incubated with anti-Ig-isotype antibodies with different fluorochromes, forming microparticle-immunoglobulin-anti-Ig-isotype complexes. After further washes, the microspheres were acquired on a multi-color flow cytometer. Appropriate flow cytometers include a FACSLyric™ or FACSCanto II™ Flow Cytometry System (Becton Dickinson, N.J.). Here a FACSCanto II was used. Values were measured as MFI ranging from 0 to 75,000 units.

Single antigen-conjugated microspheres were gated by their fluorescence characteristics and the fluorescence intensities of the fluorochromes of each type of antigen-immunoglobulin-fluorescent anti-Ig-isotype antibody complex was measured and proportionally correlated with the fluorescence intensities of the other Ig-isotypes antibody complexes against the same antigen.

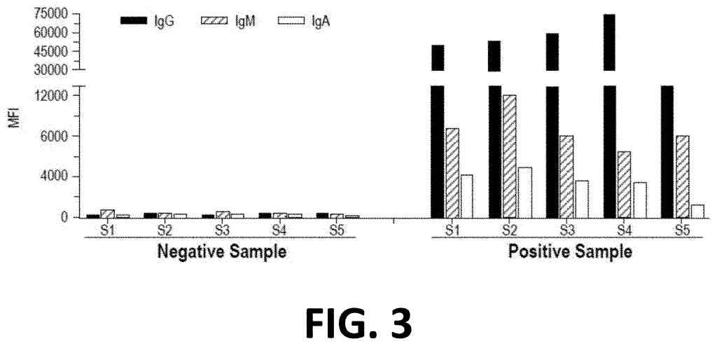

Results are presented in FIG. 3. Three individual samples are shown corresponding to three immunoglobulin isotypes each produced in response to RBD SARS-CoV-2 antigen. These results confirm that microparticle-iummunoglobulin-anti-Ig-isotype complexes form as expected under assay conditions and may be used to obtain fluorescence data that accurately reflects the expected presence or absence of test antibodies in the sample.

### Example 2

### Double-Multiplex Assay for SARS-COV-2 Exposure

Subsequent to exposure of a subject to SARS-COV-2, anti-SARS-CoV-2 antibodies may appear in the blood as a result of an immune response. Usually IgM antibodies can be detected 5-10 day after exposure or symptom onset while IgG and IgA can be detected several days later.

Double-multiplex assays as described herein can simultaneously detect the presence of three antibody isotypes (IgM, IgG and IgA) against three different SARS-CoV-2 antigens (RBD, S1, and NP) in the same well using a single test. Results are measured by a flow cytometer and presented in median fluorescence intensity (MFI, ranging from 0-262,144 MFI) data points for each antibody isotype and antigen combination.

All peripheral blood samples were collected at Stanford University using venipuncture. Seventy-nine negative samples were collected 2 years prior to the COVID-19 pandemic and 30 positive samples were collected from patients referred for testing after confirmation with SARS-CoV-2 infection using nasopharyngeal swabs submitted to RT-PCR testing. The 30 positive samples used were confirmed with an EUA-approved RT-PCR test used at the Stanford Health Center Clinical Virology Lab. The 41 convalescent samples were collected from subjects for which SARS-CoV-2 infection was confirmed using RT-PCT.

Blood was collected in standard EDTA tubes; plasma was separated and aliquoted for testing. A mixture of the identifiably labelled microsphere is combined with a test sample, allowing test antibodies in the test sample to bind to the SARS-CoV-2 antigens on the surface of the microspheres. Briefly, 5 μl of the microspheres mixture were added to each test well of a 96-well plate. Next, 50 μl of diluted test sample was added to each well and mixed. The plate was incubated at room temperature for 30 minutes allow formation of microparticle-immunoglobulin complexes. After washing the complexes three times with 150 μl of PBS buffer, 100 μl of a mixture of phycoerythrin (PE)-anti-IgG antibody, allophycocyanin (APC)-anti-IgM antibody, and fluorescein isothiocyanate (FITC)-anti-IgA antibody are added to each well allowing the formation of microparticle-immunoglobulin-anti-Ig-isotyope complexes. After washing the complexes three times with 150 μl of PBS buffer, the complexes are resuspended in 150 μl of PBS buffer and acquired on a BD FACSLyric™ flow cytometer.

Each type of identifiably labelled microsphere was distinguished and gated by its unique characteristics (size or intensity of fluorescence) and the fluorescence intensities of the multiple fluorochromes of the antigen-immunoglobulin-fluorescent anti-Ig-isotype antibody complexes were measured and proportionally correlated with the concentrations of corresponding Ig-isotypes against the same antigen. This analysis was carried out with BD FACSuite™ software, which requires at least 25 microparticle-immunoglobulin-anti-Ig-isotype complex for each type of identifiably labelled microsphere, and reads the fluorescent intensities of PE, APC, and FITS for each population of complex based on microsphere type.

Specifically, the fluorescence values are measured as mean fluorescence intensity (MFI) ranging from 0 to 250,000 units. Threshold MFI levels were established for each immunoglobulin to each antigen calculated based on mean+3 SD of known negative samples.

The double-multiplex assay provides a total of nine data points individual values for three immunoglobulin isotypes (IgM, IgG, IgA) each produced in response to three SARS-CoV-2 antigens (RBD, S1 and NP). The values were measured as median fluorescence intensity (MFI) ranging from 0 to 262,144 units. Thresholds were established for each immunoglobulin to each antigen calculated based on mean+3 SD of known negative samples.

Data points for the test samples are provided in Tables 1-3. In Tables 1-3, NPA % designates negative predictive value and PPA % designates positive predictive value.

TABLE 1
Data Points for Negative Group

| | S1 | | | RBD | | | NP | | |
|---|---|---|---|---|---|---|---|---|---|
| Sample | IgG | IgM | IgA | IgG | IgM | IgA | IgG | IgM | IgA |
| R00428 | 49 | 39 | 161 | 77 | 83 | 103 | 927 | 1233 | 1479 |
| R00205 | 54 | 37 | 161 | 128 | 109 | 131 | 1963 | 614 | 413 |
| R00408 | 51 | 26 | 158 | 179 | 103 | 104 | 2419 | 680 | 262 |
| R00410 | 63 | 37 | 167 | 180 | 126 | 113 | 1775 | 284 | 1452 |
| R00411 | 253 | 33 | 170 | 80 | 56 | 91 | 2182 | 626 | 476 |
| R00412 | 130 | 80 | 194 | 299 | 865 | 186 | 1646 | 1585 | 641 |
| R00413 | 490 | 51 | 174 | 119 | 179 | 100 | 2085 | 2310 | 2545 |
| R00416 | 214 | 42 | 177 | 98 | 153 | 93 | 1298 | 870 | 275 |
| R00418 | 384 | 30 | 156 | 155 | 170 | 166 | 3301 | 346 | 312 |
| R00419 | 84 | 83 | 169 | 120 | 181 | 101 | 1506 | 548 | 322 |
| R00197 | 272 | 39 | 170 | 101 | 98 | 99 | 1260 | 328 | 599 |
| R00198 | 56 | 26 | 159 | 409 | 80 | 98 | 1320 | 414 | 325 |
| R00199 | 82 | 66 | 164 | 118 | 85 | 91 | 2311 | 1034 | 551 |
| R00200 | 385 | 31 | 165 | 75 | 79 | 84 | 1043 | 447 | 403 |
| R00202 | 63 | 68 | 159 | 219 | 523 | 93 | 949 | 980 | 399 |
| R00203 | 418 | 29 | 181 | 58 | 72 | 90 | 698 | 1000 | 2188 |
| R00204 | 277 | 35 | 167 | 146 | 211 | 98 | 2129 | 529 | 1255 |
| R00206 | 101 | 50 | 162 | 169 | 168 | 98 | 3157 | 1157 | 287 |
| R00207 | 219 | 51 | 168 | 140 | 233 | 123 | 537 | 481 | 559 |
| R00185 | 72 | 122 | 171 | 173 | 345 | 136 | 567 | 1182 | 269 |
| R00186 | 150 | 46 | 159 | 118 | 162 | 178 | 1506 | 466 | 332 |
| R00190 | 65 | 62 | 163 | 98 | 190 | 78 | 1574 | 2830 | 388 |
| R00191 | 233 | 587 | 170 | 172 | 316 | 101 | 2140 | 1203 | 438 |
| R00192 | 729 | 172 | 174 | 163 | 228 | 100 | 2287 | 850 | 999 |
| R00193 | 108 | 22 | 182 | 68 | 46 | 92 | 2978 | 341 | 225 |
| R00194 | 89 | 37 | 156 | 56 | 98 | 82 | 1634 | 665 | 251 |
| R00195 | 76 | 40 | 165 | 117 | 64 | 103 | 1462 | 552 | 624 |
| R00196 | 128 | 84 | 162 | 99 | 283 | 123 | 2503 | 922 | 271 |
| R00442 | 207 | 63 | 169 | 135 | 248 | 106 | 3408 | 1373 | 1161 |
| R00443 | 224 | 33 | 162 | 135 | 179 | 104 | 5419 | 491 | 536 |
| R00444 | 96 | 30 | 164 | 1976 | 130 | 91 | 2635 | 582 | 353 |
| R00445 | 251 | 35 | 180 | 73 | 79 | 98 | 2243 | 624 | 1645 |
| R00446 | 61 | 30 | 161 | 79 | 96 | 98 | 2463 | 1099 | 912 |
| R00447 | 115 | 69 | 172 | 152 | 122 | 667 | 3430 | 583 | 873 |
| R00448 | 74 | 62 | 172 | 95 | 123 | 91 | 1439 | 1260 | 487 |
| R00451 | 87 | 55 | 167 | 164 | 170 | 102 | 5359 | 1708 | 396 |
| R00452 | 55 | 33 | 155 | 101 | 196 | 83 | 1478 | 531 | 2969 |
| R00429 | 61 | 20 | 171 | 1933 | 73 | 94 | 2073 | 490 | 247 |
| R00430 | 72 | 64 | 171 | 99 | 94 | 87 | 3986 | 1320 | 422 |
| R00431 | 111 | 37 | 163 | 48 | 80 | 77 | 2348 | 1674 | 230 |
| R00432 | 71 | 78 | 162 | 133 | 156 | 84 | 7409 | 1667 | 1617 |
| R00433 | 56 | 47 | 158 | 149 | 283 | 86 | 716 | 717 | 203 |
| R00435 | 43 | 25 | 157 | 59 | 190 | 84 | 1301 | 322 | 274 |
| R00437 | 65 | 55 | 153 | 71 | 96 | 76 | 1419 | 571 | 218 |
| R00440 | 57 | 28 | 162 | 75 | 73 | 91 | 1109 | 734 | 225 |
| R00441 | 60 | 26 | 159 | 119 | 164 | 93 | 1379 | 627 | 364 |
| R00420 | 65 | 29 | 165 | 112 | 69 | 146 | 1648 | 550 | 2143 |
| R00421 | 59 | 32 | 159 | 92 | 157 | 98 | 1851 | 866 | 207 |
| R00422 | 59 | 26 | 158 | 118 | 79 | 152 | 3538 | 455 | 471 |
| R00423 | 44 | 33 | 156 | 85 | 135 | 83 | 16523 | 436 | 551 |
| R00424 | 46 | 21 | 157 | 310 | 55 | 94 | 3777 | 380 | 643 |
| R00425 | 92 | 40 | 207 | 124 | 135 | 119 | 2292 | 946 | 1123 |
| R00426 | 331 | 38 | 167 | 113 | 132 | 133 | 1609 | 581 | 428 |
| R00427 | 63 | 26 | 158 | 140 | 122 | 104 | 3510 | 554 | 485 |
| R00403 | 62 | 41 | 159 | 538 | 1214 | 372 | 2562 | 829 | 902 |
| R00404 | 106 | 34 | 167 | 168 | 84 | 112 | 1167 | 673 | 605 |
| R00405 | 89 | 45 | 159 | 183 | 334 | 108 | 1473 | 501 | 271 |
| R00170 | 81 | 77 | 161 | 301 | 992 | 105 | 2041 | 937 | 469 |
| R00354 | 60 | 60 | 165 | 121 | 178 | 129 | 2921 | 819 | 444 |
| R00353 | 78 | 24 | 170 | 87 | 52 | 96 | 771 | 337 | 522 |
| R00351 | 165 | 51 | 153 | 145 | 269 | 81 | 3719 | 1175 | 187 |
| R00350 | 67 | 46 | 159 | 154 | 385 | 96 | 1012 | 772 | 225 |
| R00349 | 51 | 33 | 154 | 84 | 54 | 93 | 716 | 333 | 203 |
| R00361 | 73 | 69 | 161 | 135 | 170 | 91 | 1506 | 815 | 251 |
| R00362 | 55 | 31 | 159 | 74 | 72 | 82 | 991 | 2211 | 286 |
| R00363 | 114 | 47 | 166 | 238 | 523 | 108 | 2311 | 2092 | 516 |
| R00290 | 61 | 25 | 151 | 119 | 46 | 100 | 1587 | 401 | 617 |
| R00299 | 493 | 54 | 159 | 77 | 83 | 108 | 673 | 892 | 239 |
| R00300 | 67 | 33 | 154 | 266 | 80 | 93 | 3128 | 667 | 477 |
| R00302 | 46 | 31 | 151 | 216 | 93 | 169 | 2341 | 396 | 251 |
| R00392 | 251 | 25 | 158 | 80 | 89 | 84 | 2387 | 549 | 1470 |
| R00390 | 54 | 45 | 155 | 81 | 140 | 94 | 1951 | 924 | 260 |
| R00393 | 64 | 35 | 163 | 76 | 98 | 100 | 1779 | 527 | 612 |
| R00209 | 110 | 168 | 161 | 192 | 312 | 102 | 1707 | 2007 | 664 |
| R00210 | 118 | 63 | 189 | 347 | 153 | 126 | 1771 | 688 | 523 |
| R00357 | 105 | 148 | 156 | 391 | 630 | 104 | 1368 | 1617 | 243 |
| R00356 | 93 | 42 | 155 | 115 | 113 | 91 | 3679 | 646 | 350 |
| R00355 | 53 | 42 | 156 | 100 | 208 | 107 | 2693 | 807 | 386 |
| R00364 | 96 | 38 | 168 | 118 | 128 | 103 | 1799 | 1002 | 2717 |
| Mean + 3SD | 507 | 257 | 193 | 1087 | 797 | 334 | 8295 | 2413 | 2462 |
| NPA % | 99% | 99% | 97% | 97% | 96% | 97% | 99% | 99% | 96% |

TABLE 2
Data Points for Positive Group

| | S1 | | | RBD | | | NP | | |
|---|---|---|---|---|---|---|---|---|---|
| Sample | IgG | IgM | IgA | IgG | IgM | IgA | IgG | IgM | IgA |
| 29 | 8244 | 777 | 501 | 39381 | 10624 | 5422 | 58367 | 2758 | 16448 |
| 113 | 6086 | 330 | 643 | 45722 | 3508 | 3191 | 56022 | 2411 | 14546 |
| 125 | 6882 | 413 | 374 | 44524 | 7550 | 4535 | 60809 | 2812 | 6439 |
| 130 | 1928 | 305 | 249 | 40074 | 5059 | 5252 | 56958 | 5780 | 8054 |
| 135 | 6234 | 365 | 347 | 43692 | 7444 | 4529 | 59770 | 2819 | 6302 |
| 211 | 1538 | 369 | 219 | 29740 | 8503 | 1561 | 51424 | 2970 | 12135 |
| 314 | 5396 | 147 | 323 | 45435 | 1440 | 4315 | 60384 | 676 | 7644 |
| 331 | 69 | 67 | 193 | 293 | 425 | 141 | 6440 | 2106 | 1231 |
| 534 | 2574 | 667 | 337 | 36134 | 3669 | 7394 | 37595 | 4445 | 32163 |
| 535 | 6615 | 165 | 337 | 47448 | 1466 | 4028 | 63570 | 722 | 8206 |
| 609 | 3696 | 234 | 410 | 34323 | 20753 | 8924 | 36278 | 13920 | 46974 |
| 618 | 10765 | 680 | 1839 | 36756 | 6431 | 11610 | 32720 | 3233 | 54603 |
| 715 | 5702 | 226 | 548 | 36167 | 11732 | 11483 | 36067 | 17840 | 29542 |
| 722 | 7577 | 423 | 564 | 38673 | 16121 | 3220 | 40657 | 20731 | 7037 |
| 736 | 1311 | 500 | 294 | 29516 | 8930 | 4726 | 20390 | 22781 | 43952 |
| 752 | 563 | 66 | 178 | 24149 | 2718 | 1776 | 38777 | 20359 | 9057 |
| 771 | 3931 | 105 | 232 | 42141 | 1212 | 2227 | 60605 | 714 | 5578 |
| 951 | 16070 | 439 | 1749 | 44656 | 4643 | 9362 | 53191 | 2311 | 34438 |
| 953 | 4950 | 413 | 328 | 35910 | 6460 | 8794 | 63188 | 4246 | 4071 |
| 954 | 8214 | 206 | 525 | 38076 | 13158 | 7908 | 35669 | 20562 | 22475 |
| 957 | 7077 | 1510 | 539 | 34035 | 9704 | 6733 | 56730 | 1599 | 10518 |
| 958 | 12063 | 303 | 348 | 45855 | 4902 | 2676 | 60435 | 2336 | 6073 |
| 965 | 285 | 233 | 206 | 13224 | 6078 | 5197 | 29848 | 10070 | 10221 |
| 1003 | 5826 | 3502 | 2578 | 26151 | 24259 | 5826 | 23043 | 24850 | 35251 |
| 1025 | 3628 | 72 | 214 | 40086 | 1117 | 1224 | 60932 | 721 | 4309 |
| 1222 | 9283 | 320 | 484 | 33262 | 23839 | 6555 | 24976 | 34767 | 19231 |
| 1225 | 16940 | 772 | 1295 | 46727 | 3929 | 5120 | 59624 | 2871 | 14941 |
| 1226 | 593 | 130 | 220 | 15922 | 5348 | 5072 | 31133 | 9780 | 5950 |
| 1228 | 3605 | 301 | 233 | 36298 | 4347 | 5780 | 55886 | 4519 | 2610 |
| 1361 | 3884 | 42 | 243 | 34611 | 779 | 879 | 54845 | 641 | 3374 |
| Mean + 3SD | 507 | 257 | 193 | 1087 | 797 | 334 | 8295 | 2413 | 2462 |
| PPA % | 93% | 60% | 97% | 97% | 93% | 97% | 97% | 67% | 97% |

TABLE 3
Data Points for Positive Convalescent Group

| | S1 | | | RBD | | | NP | | |
|---|---|---|---|---|---|---|---|---|---|
| Sample | IgG | IgM | IgA | IgG | IgM | IgA | IgG | IgM | IgA |
| W070520610194 | 1163 | 66 | 201 | 17501 | 2418 | 1326 | 17568 | 837 | 488 |
| W070520610195 | 6272 | 213 | 625 | 37338 | 7341 | 6732 | 50467 | 4036 | 6528 |
| W070520610197 | 5654 | 5908 | 299 | 33870 | 4400 | 1920 | 42003 | 4724 | 1094 |
| W070520610198 | 2865 | 155 | 199 | 25764 | 6004 | 783 | 54675 | 4497 | 1459 |
| W070520610199 | 831 | 56 | 202 | 13587 | 1551 | 2210 | 40603 | 679 | 973 |
| W070520610201 | 5508 | 105 | 251 | 31302 | 3236 | 4430 | 58913 | 706 | 1088 |
| W070520610202 | 1462 | 30 | 159 | 26324 | 397 | 371 | 36387 | 214 | 3397 |
| W070520610203 | 758 | 269 | 242 | 11252 | 25752 | 1811 | 47931 | 1710 | 5545 |
| W070520610204 | 1629 | 169 | 174 | 14469 | 10612 | 575 | 60934 | 8277 | 1588 |
| W070520610205 | 145 | 67 | 159 | 1789 | 553 | 393 | 14350 | 1127 | 316 |
| W070520610206 | 511 | 95 | 168 | 10557 | 2651 | 565 | 23601 | 765 | 541 |
| W070520610207 | 214 | 202 | 168 | 7607 | 1881 | 331 | 18589 | 2292 | 397 |
| W070520610208 | 4324 | 77 | 173 | 17578 | 1069 | 202 | 47369 | 707 | 491 |
| W070520610209 | 1313 | 122 | 216 | 21004 | 2082 | 1084 | 49715 | 4381 | 1434 |
| W070520610210 | 669 | 199 | 175 | 16405 | 3177 | 793 | 24270 | 1232 | 1437 |
| W070520610211 | 1804 | 193 | 210 | 27722 | 3260 | 1109 | 55305 | 879 | 1508 |
| W070520610212 | 345 | 29 | 161 | 7620 | 1396 | 249 | 35339 | 2092 | 768 |
| W070520610213 | 3131 | 73 | 1059 | 23721 | 2978 | 789 | 50483 | 5755 | 3510 |
| W070520610214 | 347 | 46 | 160 | 8246 | 683 | 334 | 25930 | 498 | 404 |
| W070520610215 | 367 | 51 | 163 | 9900 | 4496 | 957 | 47638 | 508 | 1123 |
| W070520610216 | 4362 | 208 | 266 | 37932 | 3938 | 2639 | 57021 | 2195 | 10364 |
| W070520610217 | 4172 | 4549 | 255 | 25370 | 3653 | 1664 | 32825 | 4299 | 850 |
| W070520610218 | 1436 | 42 | 160 | 18267 | 1577 | 352 | 50083 | 1389 | 2003 |
| W070520610219 | 369 | 18 | 171 | 16785 | 206 | 233 | 49414 | 2105 | 1556 |
| W070520610220 | 6246 | 130 | 227 | 37717 | 2424 | 1196 | 53440 | 1613 | 1173 |
| W070520610221 | 232 | 79 | 157 | 2091 | 778 | 1099 | 21340 | 2215 | 1019 |
| W070520610222 | 2084 | 155 | 191 | 24441 | 3710 | 2661 | 53390 | 3278 | 1168 |
| W070520610223 | 1107 | 333 | 251 | 13696 | 2394 | 4111 | 38863 | 2181 | 976 |
| W070520610224 | 4531 | 81 | 189 | 28521 | 5985 | 562 | 41884 | 10891 | 4731 |
| W070520610226 | 3434 | 118 | 235 | 40043 | 3538 | 4507 | 43010 | 7420 | 2280 |
| W070520610227 | 5179 | 102 | 218 | 35391 | 1989 | 1066 | 50688 | 1341 | 1111 |
| W070520610228 | 812 | 50 | 172 | 11651 | 434 | 430 | 17244 | 1275 | 1217 |
| W070520610229 | 1064 | 73 | 163 | 17585 | 3800 | 358 | 40457 | 8755 | 763 |
| W070520610230 | 1060 | 22 | 158 | 20523 | 202 | 256 | 30514 | 174 | 1638 |
| W070520610231 | 1744 | 145 | 203 | 24297 | 2241 | 990 | 51842 | 858 | 1477 |
| W070520610234 | 848 | 117 | 181 | 9036 | 19259 | 1438 | 47266 | 2036 | 613 |
| W070520610235 | 1838 | 123 | 181 | 17995 | 2953 | 2089 | 43131 | 1235 | 2416 |
| W070520610236 | 3509 | 3470 | 232 | 20127 | 2961 | 1358 | 26453 | 3682 | 648 |
| W070520610237 | 414 | 249 | 179 | 6174 | 3001 | 1251 | 29423 | 2156 | 1280 |
| W070520213255 | 3581 | 179 | 203 | 35078 | 1720 | 580 | 51581 | 1989 | 2118 |
| W070520610238 | 557 | 87 | 161 | 9184 | 3526 | 400 | 28645 | 1098 | 551 |
| Mean + 3SD | 507 | 257 | 193 | 1087 | 797 | 334 | 8295 | 2413 | 2462 |
| PPA % | 80% | 12% | 46% | 100% | 83% | 88% | 100% | 29% | 15% |

Results for each type of data point were tallied and sensitivity, specificity, concordance, and predictive values were calculated, as presented in Tables 4-12. Results in the tables are mean+3 SD.

TABLE 4
S1 IgG

Predicate PCR Result

| Assay Result | + | − |
|---|---|---|
| + | 61 | 1 |
| − | 10 | 78 |

There were 139 concordant results and 11 discordant results from 150 test samples. Additional indicators of accuracy were as follows:

Sensitivity: 85.9%

Specificity: 98.7%

Concordance (Correlation): 0.927

Positive Predictive Value: 98.4%

Negative Predictive Value: 88.6%

False Positive Rate: 1.6%

False Negative Rate: 11.4%

TABLE 5
S1 IgM

Predicate PCR Result

| Assay Result | + | − |
|---|---|---|
| + | 23 | 1 |
| − | 48 | 78 |

There were 101 concordant results and 49 discordant results from 150 test samples. Additional indicators of accuracy were as follows:

Sensitivity: 32.4%

Specificity: 98.7%

Concordance (Correlation): 0.673

Positive Predictive Value: 95.8%

Negative Predictive Value: 61.9%

False Positive Rate: 4.2%

False Negative Rate: 38.1%.

TABLE 6
S1 IgA

Predicate PCR Result

| Assay Result | + | − |
|---|---|---|
| + | 48 | 2 |
| − | 23 | 77 |

There were 125 concordant results and 25 discordant results from 150 test samples. Additional indicators of accuracy were as follows:

Sensitivity: 67.6%

Specificity: 97.5%

Concordance (Correlation): 0.833

Positive Predictive Value: 96.0%

Negative Predictive Value: 77.0%

False Positive Rate: 4.0%

False Negative Rate: 23.0%

TABLE 7
RBD IgG

Predicate PCR Result

| Assay Result | + | − |
|---|---|---|
| + | 70 | 2 |
| − | 1 | 77 |

There were 147 concordant results and 3 discordant results from 150 test samples. Additional indicators of accuracy were as follows:

Sensitivity: 98.6%

Specificity: 97.5%

Concordance (Correlation): 0.980

Positive Predictive Value: 97.2%

Negative Predictive Value: 98.7%

False Positive Rate: 2.8%

False Negative Rate: 1.3%

TABLE 8
RBD IgM

Predicate PCR Result

| Assay Result | + | − |
|---|---|---|
| + | 62 | 3 |
| − | 9 | 76 |

There were 138 concordant results and 12 discordant results from 150 test samples. Additional indicators of accuracy were as follows:

Sensitivity: 87.3%

Specificity: 96.2%

Concordance (Correlation): 0.920

Positive Predictive Value: 95.4%

Negative Predictive Value: 89.4%

False Positive Rate: 4.6%

False Negative Rate: 10.6%

TABLE 9
RBD IgA

Predicate PCR Result

| Assay Result | + | − |
|---|---|---|
| + | 65 | 2 |
| − | 6 | 77 |

There were 142 concordant results and 8 discordant results from 150 test samples. Additional indicators of accuracy were as follows:

Sensitivity: 91.5%

Specificity: 97.5%

Concordance (Correlation): 0.947

Positive Predictive Value: 97.0%

Negative Predictive Value: 92.8%

False Positive Rate: 3.0%

False Negative Rate: 7.2%

TABLE 10
NP IgG

Predicate PCR Result

| Assay Result | + | − |
|---|---|---|
| + | 70 | 1 |
| − | 1 | 78 |

There were 148 concordant results and 2 discordant results from 150 test samples. Additional indicators of accuracy were as follows:

Sensitivity: 98.6%

Specificity: 98.7%

Concordance (Correlation): 0.987

Positive Predictive Value: 98.6%

Negative Predictive Value: 98.7%

False Positive Rate: 1.4%

False Negative Rate: 1.3%

TABLE 11
NP IgM

Predicate PCR Result

| Assay Result | + | − |
|---|---|---|
| + | 33 | 1 |
| − | 38 | 78 |

There were 111 concordant results and 39 discordant results from 150 test samples. Additional indicators of accuracy were as follows:

Sensitivity: 46.5%

Specificity: 98.7%

Concordance (Correlation): 0.740

Positive Predictive Value: 97.1%

Negative Predictive Value: 67.2%

False Positive Rate: 2.9%

False Negative Rate: 32.8%

TABLE 12
NP IgA

Predicate PCR Result

| Assay Result | + | − |
|---|---|---|
| + | 35 | 3 |
| − | 36 | 76 |

There were 111 concordant results and 39 discordant results from 150 test samples. Additional indicators of accuracy were as follows:

Sensitivity: 49.3%

Specificity: 96.2%

Concordance (Correlation): 0.740

Positive Predictive Value: 92.1%

Negative Predictive Value: 67.9%

False Positive Rate: 7.9%

False Negative Rate: 32.1%

The data points were further used to determine if the test sample was positive or negative for a particular anti-SARS-CoV-2 antibody of a given isotype. This determination was based on concordance of the data points for the three separate antigens. If there was a positive result for a particular isotype for two antigens, then the test sample was determined to be positive for antibodies of that isotype against SARS-Co-V2. If there was a negative result for a particular isotype for two antigens, then the test sample was determined to be negative for antibodies for that isotype against SARS-Co-V2. Thus, measurement of levels of immunoglobulin isotypes against three separate viral antigens enhanced the specificity because chances for cross-reactivity against three antigens is presumed to be less than one antigen. Sensitivity was enhanced because while levels of an immunoglobulin isotype may be low for one antigen, these levels may be higher for the other two antigens, thus reducing the chances of false negatives. This method has the advantage of allowing enhancement of specificity and sensitivity together rather than one at the expense of the other.

An evaluation of the overall specificity and sensitivity of the double-multiplex assay for COVID-19 is shown in Table 13.

TABLE 13
SARS-CoV-2 Exposure

Predicate PCR Result

| Assay Result | + | − |
|---|---|---|
| + | 70 | 0 |
| − | 1 | 79 |

There were 150 concordant results and 1 discordant results from 150 test samples. Additional indicators of accuracy were as follows:

Sensitivity: 98.6%

Specificity: 100.0%

Concordance (Correlation): 0.993

Positive Predictive Value: 100.0%

Negative Predictive Value: 98.8%

False Positive Rate: 0.0%

False Negative Rate: 1.3%

The overall sensitivity of the double-multiplex assay was high because the assay measured levels of three different immunoglobulin isotypes against three different antigens. Specificity was further increased by requiring MFI values above the cut point for at least one immunoglobulin isotype against each of at least two antigens in order to identify a result as positive for any individual patient. Because of the high sensitivity and specificity of the method, an equivocal range was not required.

Negative controls for the double-multiplex assay were established for each of the nine possible types of data points. Serum samples with MFI results of <50% of the threshold MFI level for each type of data point were. Samples with the lowest possible MFI for each type of data point were used. A minimum of 5 samples were used to create the pool. The samples were mixed carefully, avoiding foam formation. Aliquots of at least 200 μL were prepared from this serum pool and stored frozen at −20 degrees Celsius or colder. These aliquots were used to perform regular quality control. Westgard rules were used to establish the control ranges and monitor assay control. Once thawed, aliquots were stable for 1 week, if stored refrigerated.

Positive controls for the double-multiplex assay were also established for each of the nine possible types of data points. Serum samples with MFI results >5 times the cut-off level for each of the 9 reportable results were pooled. A minimum of 5 samples were used to create the pool. The samples were mixed carefully, avoiding foam formation. The assay pool was diluted, if necessary, by adding pooled negative serum (for pooling criterion see negative control above) to obtain MFI values between 3 to 20 times the threshold for each of the nine types of data points. Aliquots of at least 200 μL from this sample pool were prepared and stored frozen at −20 degrees Celsius or colder. Westgard rules were used to establish the control ranges and monitor assay control. Once thawed, aliquots were stable for 1 week, if stored refrigerated.

The identifiably labeled microspheres and test buffers are manufactured based on standard operation protocols and QC system.

Microparticles, antigens, and secondary antibodies underwent stability testing. Tables 1-4 below show the results of stability testing. Based on these results, no loss of activity was observed at after storage for up to four months at 4 degrees Celsius or −80 degrees Celsius

### Example 3

### Comparative Analysis: Specificity and Sensitivity of Double-Multiplex Technology Compared with ELISA

ELISA is a plate-based technique commonly used to detect and quantify antiviral antibodies. The method utilizes viral protein antigens coated on plastic microtiter plates to capture antiviral antibodies in a sample, which may be derived from a number of bodily fluids, including blood, serum, and sputum, among others. The sample is left in contact with the coated antigen to allow relevant antibodies to bind, after which the plate is washed several times. Captured antibodies are detected by secondary species-specific antibodies complexed with a reporter enzyme that, when provided with the appropriate substrate, produces a measurable output.

The sensitivity and specificity of the double-multiplex assay of Example 2 was compared with that of an ELISA.

Ig isotypes (IgG, IgM, and IgA) against two SARS-CoV-2 antigens, RBD, and NP, were detected using a conventional ELISA format, in which there is no multiplexing and only a single antigen and anti-Ig-isotype are present in each sample. Results are presented in Table 14. − group, n=70, + group, n=30. Indicated percentages are predictive value of results. Bold and italics indicate values that do not meet FDA requirements for an Emergency Use Authorization (EUA). PPA designates positive predictive value and NPA designates negative predictive value.

TABLE 14
ELISA Results

| | S1 | | | RBD | | | NP | | |
|---|---|---|---|---|---|---|---|---|---|
| | IgG | IgM | IgA | IgG | IgM | IgA | IgG | IgM | IgA |
| −Group | NA | NA | NA | 99% | 99% | 99% | 97% | 97% | 97% |
| (PPA) | | | | | | | | | |
| +group | NA | NA | NA | 97% | 43% | 83% | 23% | 25% | 97% |
| (NPA) | | | | | | | | | |

As these results indicate, ELISA-based testing may not produce sufficiently accurate results, particularly with respect to IgM antibodies likely to be present soon after exposure to SARS-CoV-2.

Results from Example 2 are provided in condensed form in Table 15. − group, n=70, + group, n=30, convalescent patients group (C group), n=41. Indicated percentages are predictive value of results. Bold and italics indicate values that do not meet FDA requirements for an Emergency Use Authorization (EUA) in the context of the + group and − group.

TABLE 15
Double-Multiplex Assay Results

| | S1 | | | RBD | | | NP | | |
|---|---|---|---|---|---|---|---|---|---|
| | IgG | IgM | IgA | IgG | IgM | IgA | IgG | IgM | IgA |
| −Group | 99% | 99% | 97% | 97% | 96% | 97% | 99% | 99% | 96% |
| (NPA) | | | | | | | | | |
| +group | 93% | 60% | 97% | 97% | 93% | 97% | 97% | 67% | 97% |
| (PPA) | | | | | | | | | |
| C group | 80% | 12% | 46% | 100% | 83% | 88% | 100% | 29% | 15% |
| (PPA) | | | | | | | | | |

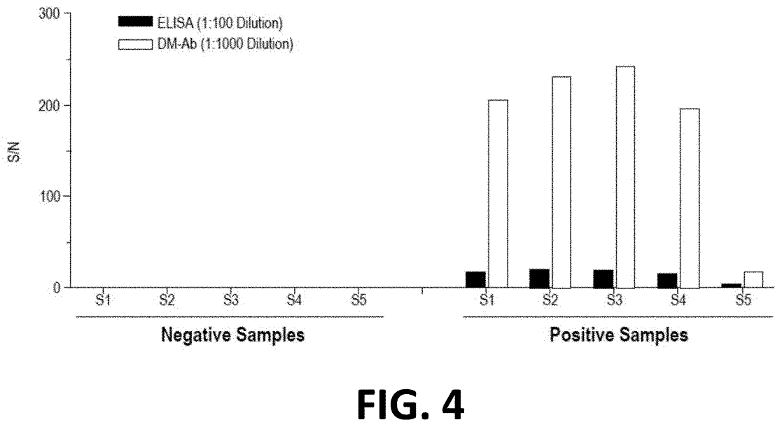

A comparison of assay sensitives is presented in FIG. 4.

As these results indicate, a double-multiplex assay of the present disclosure can detect antibodies against SARS-CoV-2 antigens in positive samples at least as well as an ELISA. In addition, by separately detecting multiple immunoglobulin isotypes against multiple antigens in a single assay, the assay is more likely to still yield a positive result for patients who have been exposed to SARS-CoV-2, particularly convalescent patients, than an ELISA.

### Example 4

### Double-Multiplex Assay in Vaccinated Subjects

A double-multiplex assay as set forth in Example 2 was conducted using additional patient samples from subjects before and three weeks after vaccination for SARS-CoV-2. The resulting data is provided in Tables 16 and 17. The data further confirms the specificity and sensitivity of the assay and demonstrates that it can detect antibodies in vaccinated subjects.

TABLE 16
Pre- Vaccination

| | S1 | | | RBD | | | NP | | |
|---|---|---|---|---|---|---|---|---|---|
| Sample | IgG | IgM | IgA | IgG | IgM | IgA | IgG | IgM | IgA |
| 1 | 131 | 273 | 358 | 932 | 2855 | 457 | 2200 | 12228 | 536 |
| 2 | 171 | 143 | 338 | 1877 | 6052 | 226 | 2713 | 7516 | 838 |
| 3 | 182 | 209 | 341 | 1026 | 3186 | 264 | 2019 | 6498 | 595 |
| 4 | 118 | 29 | 316 | 206 | 214 | 181 | 1527 | 649 | 462 |
| 5 | 121 | 50 | 320 | 834 | 2088 | 328 | 1212 | 2829 | 659 |
| 6 | 151 | 200 | 319 | 222 | 765 | 160 | 963 | 2850 | 548 |
| 7 | 154 | 59 | 323 | 312 | 1224 | 128 | 7378 | 1717 | 1805 |
| 8 | 131 | 98 | 313 | 1423 | 2018 | 752 | 2506 | 4866 | 1286 |
| 9 | 170 | 200 | 351 | 311 | 927 | 148 | 4889 | 5096 | 3568 |
| 10 | 126 | 50 | 388 | 308 | 420 | 620 | 12236 | 44547 | 650 |
| 11 | 99 | 49 | 360 | 261 | 328 | 286 | 502 | 914 | 707 |
| Cutoff | 364 | 430 | 386 | 1527 | 1818 | 297 | 5070 | 6717 | 1936 |

TABLE 17
Post- Vaccination

| | S1 | | | RBD | | | NP | | |
|---|---|---|---|---|---|---|---|---|---|
| Sample | IgG | IgM | IgA | IgG | IgM | IgA | IgG | IgM | IgA |
| 1 | 59792 | 3220 | 1146 | 77256 | 7145 | 2340 | 4149 | 21665 | 643 |
| 2 | 45818 | 2160 | 1455 | 71815 | 10050 | 2552 | 3213 | 7942 | 999 |
| 3 | 43871 | 1324 | 799 | 58126 | 4080 | 1631 | 1395 | 4030 | 563 |
| 4 | 59350 | 4226 | 1114 | 89099 | 8125 | 1795 | 2128 | 680 | 374 |
| 5 | 67857 | 6778 | 10286 | 93544 | 9571 | 15419 | 2720 | 8578 | 580 |
| 6 | 52856 | 1838 | 1309 | 79253 | 3261 | 2190 | 2100 | 6308 | 647 |
| 7 | 78691 | 3240 | 4031 | 108837 | 6055 | 4089 | 10247 | 3042 | 3377 |
| 8 | 101078 | 3348 | 1253 | 130015 | 6411 | 2074 | 2572 | 4756 | 1474 |
| 9 | 72728 | 2610 | 2277 | 104770 | 4136 | 3810 | 3418 | 2809 | 485 |
| 10 | 39717 | 1438 | 1275 | 58602 | 2984 | 2151 | 15227 | 40674 | 829 |
| 11 | 47439 | 3070 | 6649 | 71935 | 5672 | 11551 | 945 | 1117 | 857 |
| Cutoff | 364 | 430 | 386 | 1527 | 1818 | 297 | 5070 | 6717 | 1936 |

This data demonstrates the ability of the double-multiplex assay to detect antibody production in vaccinated subjects.

### Example 5

### SARS-COV-2 Assay Report

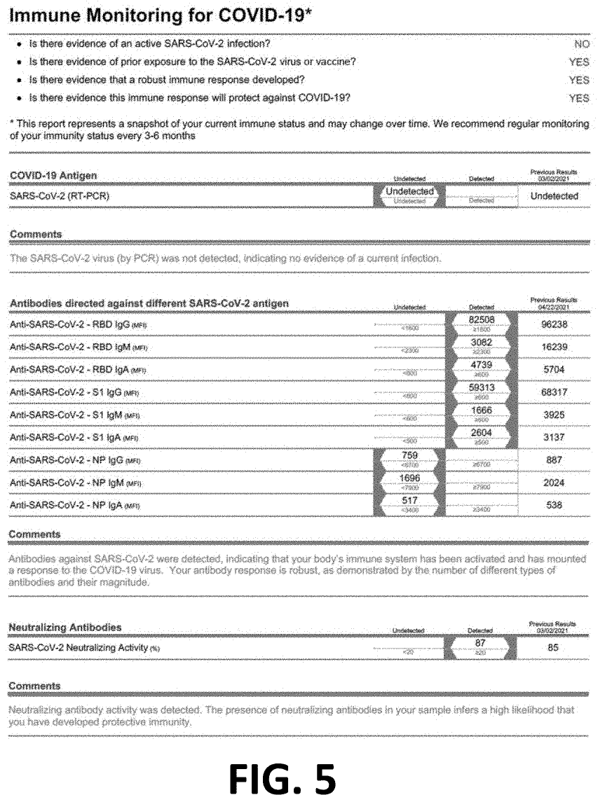

FIG. 5 is an exemplary report from a double-multiplex assay for antibodies against SARS-CoV-2. The report may be used for providing a diagnosis to the subject who provided the test sample. The example report provides data points associated with the test sample in the form of measurements in in the “Antibodies directed against different SARS-CoV-2 antigen” portion of the report under the “Undetected” and “Detected” columns. Thresholds for positivity or negativity of these data points are also indicated. The type of data point (e.g. Anti-SARS-CoV-2 RBD IgG) and the type of measurement (MFI) are also provided to assist with understanding and identifying the included data points.

The exemplary report further provides information, in the form of a positivity (“yes”) or negativity (“no” indicator) for two test sample properties, “Is there evidence of prior exposure to the SARS-CoV-2 virus or vaccine?” and “Is there evidence that a robust response developed?” These test sample properties are determined through reference to the data points. The exemplary report further includes diagnostic information in the form of “Comments.” Such diagnostic information may be used by the subject directly, or in combination with further advice from a medical professional.

Additional information contained in the exemplary test report may be of further use in providing a diagnosis or to derive further test sample properties. For instance, the “Previous Results” provided may be compared against the current results to determine additional diagnostic information or test sample properties.

In the example of FIG. 5, results from additional tests, in particular, an RT-PCR test and a neutralizing antibody test, are also provided and may be combined with results from the double-multiplex assay to provide diagnostic information to the subject.

The various embodiments described above can be combined to provide further embodiments. All of the U.S. patents, U.S. patent application publications, U.S. patent applications, foreign patents, foreign patent applications and non-patent publications referred to in this specification or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A double-multiplex assay method of detecting at least two isotypes of antibodies against at least two antigens in a test sample, the method comprising:
a) combining a test sample containing test antibodies with a mixture of at least two types of identifiably labelled microparticles, wherein each type of identifiably labelled microparticles is conjugated to a different antigen, to form microparticle-immunoglobulin complexes with test antibodies that specifically bind the antigens;
b) combining the microparticle-immunoglobulin complexes with detectably labelled anti-Ig-isotype antibodies against at least two different immunoglobulin isotypes to form microparticle-immunoglobulin-anti-Ig-isotype complexes;
c) detecting identifiably labelled microparticle type and anti-Ig-isotype antibody type for the microparticle-immunoglobulin-anti-Ig-isotype complexes to generate detection data;
d) combining or analyzing detection data to generate at least four distinct data points, each data point corresponding to a different combination of test antibody isotype and antigen specificity;
e) using the data points to determine a test sample property.

2. The method of claim 1, wherein the different antigens are from a single biological source and the test sample property is whether the subject is positive or negative for antibodies against the biological source.

3. The method of claim 1, wherein at least three different antigens are conjugated to at least three types of identifiably labelled microparticles and detectably labelled anti-Ig-isotype antibodies against at against least three different immunoglobulin isotypes are used to generate at least nine distinct types of data points.

4. The method of claim 1, wherein the test sample is from a human subject.

5. The method of claim 1, wherein the test sample has a volume of 0.1-20.0 μL.

6. The method of claim 1, wherein the test sample is whole blood, serum, plasma, nasal secretions, sputum, bronchial lavage, urine, stool, or saliva.

7. The method of claim 1, wherein the biological sample is whole blood, serum, or plasma.

8. The method of claim 7, wherein the whole blood, serum, or plasma is obtained by finger-stick.

9. The method of claim 1, wherein the test sample is diluted prior to combining with mixture of at least two types of identifiably labelled microparticles.

10. The method of claim 9, wherein the diluted biological sample has a volume of 20-50 μl.

11. The method of claim 1, wherein the identifiably labelled microparticles are microspheres.

12. The method of claim 1, wherein the microparticles have a cross-section that is from 0.001 μm to 1000 μm in length.

13. The method of claim 1, wherein the identifiably labelled microparticles are identifiable by size, magnetic properties, fluorescence, ultraviolet-excited fluorescence wavelength, violet-excited fluorescence wavelength, fluorescence intensity, metal isotopes, or any combination thereof.

14. The method of claim 1, wherein the detectably labelled anti-Ig-isotype antibodies are identifiable by fluorescence properties, luminescent properties, or colorimetric properties or any combinations thereof.

15. The method of claim 1, wherein the anti-Ig-isotype antibodies comprise antibodies against IgG, IgM, IgA, or any combinations thereof.

16. The method of claim 15, wherein the antigens are from a virus, bacteria, transplanted organ or tissue, tumor, or cancer.

17. The method of claim 1, wherein the anti-Ig-isotype antibodies comprise antibodies against IgG subtypes.

18. The method of claim 17, wherein the antigens are from a virus, bacteria, transplanted organ or tissue, tumor, or cancer.

19. The method of claim 1, wherein the anti-Ig-isotype antibodies comprise antibodies against IgE subtypes.

20. The method of claim 19, wherein the antigens are from an allergen.

21. The method of claim 1, wherein the microparticle-immunoglobulin complexes are combined with a mixture of the detectably labelled anti-Ig-isotype antibodies.

22. The method of claim 1, wherein the microparticle-immunoglobulin complexes are combined with each type of the detectably labelled anti-Ig-isotype antibodies separately in sequential steps.

23. The method of claim 1, wherein the detecting step is carried out using flow cytometry or mass cytometry.

24. The method of claim 1, wherein steps a)-c) are carried out in a period of time of about 30 minutes to 3 hours.

25. The method of claim 1, further comprising determining at least one indicator of accuracy for each data point, wherein the indicator of accuracy is sensitivity, specificity, concordance (correlation), positive predictive value, negative predictive value, false positive rate, or false negative rate.

26. The method of claim 1, wherein the test sample property is positivity or negativity of the test sample for test antibodies of a specific antibody isotype, and positivity or negativity is determined by concordance of data points for the antibody isotype against all antigens.

27. The method of claim 26, further comprising determining at least one indicator of accuracy for the test sample property, wherein the indicator of accuracy is sensitivity, specificity, concordance (correlation), positive predictive value, negative predictive value, false positive rate, or false negative rate.

28. The method of claim 27, wherein the specificity of the test sample property is increased without a decrease in sensitivity as compared to a corresponding assay that uses only a single type of data point to determine the test sample property.

29. The method of claim 28, wherein the specificity is increased at least ten fold as compared to a corresponding assay that uses only a single type of data point to determine the test sample property.

30. The method of claim 1, wherein the test sample property is positivity or negativity of the test sample for test antibodies against a specific antigen, and positivity or negativity is determined by concordance of data points for antibodies against the antigen for all antibody isotypes.

31. The method of claim 30, further comprising determining at least one indicator of accuracy for the test sample property, wherein the indicator of accuracy is sensitivity, specificity, concordance (correlation), positive predictive value, negative predictive value, false positive rate, or false negative rate.

32. The method of claim 31, wherein the specificity of the test sample property is increased without a decrease in sensitivity as compared to a corresponding assay that uses only a single type of data point to determine the test sample property.

33. The method of claim 32, wherein the specificity is increased at least ten fold as compared to a corresponding assay that uses only a single type of data point to determine the test sample property.

34. A system for double-multiplexed assay of a test sample for at least two isotypes of antibodies against at least two antigens, the system comprising:
a) at least two types of identifiably labelled microparticles conjugated to at least two antigens, wherein each type of identifiably labelled microparticle is conjugated to a different antigen;
b) at least two types of microparticle-immunoglobulin complexes, wherein each type of microparticle-immunoglobulin complex comprises an identifiably labelled microparticle conjugated to an antigen and a test antibody from the test sample specifically bound to the antigen; and
c) at least two types of microparticle-immunoglobulin-anti-Ig-isotype complexes, wherein each type of microparticle-immunoglobulin-anti-Ig-isotype complex comprises an identifiably labelled microparticle conjugated to an antigen, a test antibody from the test sample specifically bound to the antigen, and at least one detectably labelled anti-Ig-isotype antibody bound to the test antibody.

35. The system of claim 34, wherein each type of microparticle-immunoglobulin-anti-Ig-isotype complex comprises at least two types of detectably labelled anti-Ig-isotype antibodies bound to the test antibodies.

36. A kit for double-multiplexed assay of a test sample for at least two isotypes of antibodies against at least two antigens, the comprising:
a) one or more types of identifiably labelled microparticles, wherein each type of microparticle is conjugated to a different antigen; and
b) two or more types of detectably labelled anti-Ig-isotype antibodies, wherein each type of anti-Ig-isotype antibody binds a different immunoglobulin isotype or subtype.
